(19) United States
(12) Patent Application Publication
(10) Pub. No.: US 2010/0212036 A1
(43) Pub. Date: Aug. 19, 2010

(54) SELECTIVE INHIBITION OF HISTONE DEACETYLASES 1 AND 2 AS A TREATMENT FOR CARDIAC HYPERTROPHY

(76) Inventors: Eric N. Olson, Dallas, TX (US); Lawrence Melvin, Longmont, CO (US); Rusty L. Montgomery, Superior, CO (US)

Correspondence Address: FULBRIGHT & JAWORSKI L.L.P., 600 CONGRESS AVE., SUITE 2400, AUSTIN, TX, 78701, US

(21) Appl. No.: 12/589,528

(22) Filed: Sep. 29, 2009

Related U.S. Application Data
(60) Provisional application No. 61/101,547, filed on Sep. 30, 2008.

(51) Int. Cl. A01K 67/027; G01N 33/567; A01K 67/00; A61K 31/216; A61K 31/351; A61K 31/455; A61K 31/715; A61K 31/352; A61K 31/727; A61K 38/49; A61K 31/164; A61K 31/40; A61K 31/138; A61P 9/00

(52) U.S. Cl. 800/18; 435/7.21; 800/13; 800/14; 514/543; 514/460; 514/356; 514/54; 514/457; 514/56; 424/94.64; 514/620; 514/423; 514/652

(57) ABSTRACT

The present invention provides for methods of treating and preventing cardiac hypertrophy. Class I HDACs, which are known to participate in regulation of chromatin structure and gene expression, have generally been considered as pro-hypertrophic in their action. However, the present invention demonstrates that inhibition of certain Class I HDACs should be avoided in the treatment of cardiac hypertrophy, thereby pointing toward selective, and not global, inhibition of Class I HDACs. In particular, the present invention provides for selective inhibition of HDACs 1 and/or 2, and the avoidance of inhibition of HDAC3.

### PRIORITY

This application claims benefit of priority to U.S. Provisional Application Ser. No. 61/101,547, filed Sep. 30, 2008, the entire contents of which are hereby incorporated by reference.

### GOVERNMENT SUPPORT

This invention was made with government support under 5 R37 HL53351-14 awarded by the National Institutes of Health. The government has certain rights in the invention.

### BACKGROUND OF THE INVENTION

1. Field of the Invention

The present invention relates generally to the fields of developmental biology and molecular biology. More particularly, it concerns gene regulation and cellular physiology in cardiomyocytes. Specifically, the invention relates to the use of HDAC1/2 selective inhibitors to treat cardiac hypertrophy and heart failure.

2. Description of Related Art

Heart failure is a complex disorder that arises from multiple pathological insults including myocardial infarction, hypertension, and coronary artery disease. Initially, these insults are followed by an increase in cardiac mass through hypertrophy of cardiomyocytes to sustain cardiac output, however prolonged hypertrophy often results in cardiac dilatation and failure (Gardin and Lauer, 2004; Devereux et al., 2004; Okin et al., 2004). In addition to increases in cardiomyocyte size and organization of the sarcomere, the stressed myocardium also responds by transcriptionally reprogramming of the cardiomyocyte genome to a more fetal-like gene state (Hill and Olson, 2008; Olson and Schneider, 2003). Multiple signaling pathways and transcription factors have been implicated in regulating hypertrophic gene activation including GATA4, NFAT, and MEF2 (Frey and Olson, 2003).

Pharmacological inhibition of chromatin-modifying enzymes has recently emerged as an effective means of modulating pathological changes in gene expression in a variety of diseases (McKinsey and Olson, 2005). Nucleosomal histone tails can be post-translationally modified individually or in concert with other modifications to precisely control gene expression (Jenuwein and Allis, 2001). Lysine acetylation of histone tails is coupled to transcriptional activation as the loss of positive charge relaxes chromatin, allowing for the recruitment of transcriptional machinery by DNA-bound transcription factors and subsequent gene activation (Roth et al., 2001). Acetylation of lysine residues is catalyzed by histone acetyltransferases (HATs) and opposed by histone deacetylases (HDACs). HDACs remove acetyl moieties from histone tails, resulting in chromatin condensation and an overall reduction in transcriptional potential. The interplay between HATs and HDACs allows for rapid changes in gene expression in response to extrinsic or intrinsic signals and as such, has become a therapeutic target for a variety of pathological states including cancer and cardiac disease.

Studies in mice and cultured cardiomyocytes have identified both class I and class II HDACs as key regulators of cardiac growth and disease (McKinsey and Olson, 2004). Class II HDACs bind and repress MEF2 under normal physiologic conditions, however in response to hypertrophic stimuli, these HDACs become phosphorylated and exported from the nucleus allowing for the de-repression of MEF2 and other transcription factors (McKinsey et al., 2000; Zhang et al., 2002). Paradoxically, inhibition of HDACs is able to blunt the hypertrophic response, suggesting a role for class I HDACs in cardiac hypertrophy (Antos et al., 2003; Kee et al., 2006; Kong et al., 2006).

The four class I HDACs (HDAC1, HDAC2, HDAC3 and HDAC8) share extensive homology and are widely expressed (Grozinger and Schreiber, 2002), but little is known of their individual functions in vivo. Previously, the inventors showed that cardiac deletion of either HDAC1 or HDAC2 in the heart did not affect cardiac structure or function, whereas deletion of both of these HDACs resulted in perinatal lethality from cardiac arrhythmias, accompanied by dilated cardiomyopathy, and up-regulation of genes encoding skeletal muscle-specific contractile proteins and calcium channels (Montgomery et al., 2007). Using a lacZ enhancer trap allele of HDAC2, other investigators showed that HDAC2 mutant mice are viable, but resistant to pathological hypertrophy (Trivedi et al., 2007). However, further information on the role these molecules play in cardiac hypertrophy, as well as the effects of inhibiting their function, are needed.

### SUMMARY OF THE INVENTION

Thus, in accordance with the present invention, there is provided a method of treating pathologic cardiac hypertrophy and/or heart failure comprising (a) identifying a patient having pathologic cardiac hypertrophy and/or heart failure; and (b) administering to said patient a histone deacetylase inhibitor that selectively inhibits HDAC1, HDAC2, or both HDAC1 and HDAC2, over HDAC3. The inhibitor may be a heteroaryl substituted benzamide, a biaryl benzamide, optionally substituted, or an aminophenyl benzamide. Administering may comprise oral administration of said histone deacetylase inhibitor, or intravenous, transdermal, sustained release, suppository, or sublingual administration. The method may comprises administering to said patient a second therapeutic regimen, such as a β blocker, an iontrope, diuretic, ACE inhibitor, All antagonist, Ca⁺⁺-blocker, nitrate, thrombolytic, and anti-platelet. The second therapeutic regimen may be administered at the same time as said histone deacetylase inhibitor, or either before or after said histone deacetylase inhibitor. Treating may comprise improving one or more symptoms of pathologic cardiac hypertrophy and/or heart failure, such as increased exercise capacity, increased blood ejection volume, left ventricular end diastolic pressure, pulmonary capillary wedge pressure, cardiac output, cardiac index, pulmonary artery pressures, left ventricular end systolic and diastolic dimensions, left and right ventricular wall stress, or wall tension, quality of life, disease-related morbidity and mortality.

In another embodiment, there is provided a method of preventing pathologic cardiac hypertrophy and/or heart failure comprising (a) identifying a subject at risk of developing pathologic cardiac hypertrophy and/or heart failure; and (b) administering to said subject a histone deacetylase inhibitor that selectively inhibits HDAC1 and/or HDAC2 over HDAC3. The inhibitor may be a heteroaryl substituted benzamide, a biaryl benzamide, optionally substituted, or an aminophenyl benzamide. Administering may comprise oral administration of said histone deacetylase inhibitor, or intravenous, transdermal, sustained release, suppository, or sublingual administration. The subject may be at risk may exhibit one or more of long standing uncontrolled hypertension, uncorrected valvular disease, chronic angina and/or recent myocardial infarction. The method may further comprise administering to said subject a second prophylatic regimen, such as a β blocker, an iontrope, diuretic, ACE-I, All antagonist, Ca⁺⁺-blocker, nitrate, thrombolytic, and anti-platelet. The second prophylatic regimen may be administered at the same time as said histone deacetylase inhibitor, or either before or after said histone deacetylase inhibitor. Preventing may comprise preventing pathological cardiac hypertrophy from developing into heart failure.

In still another embodiment, there is provided a method of identifying an inhibitor of pathologic cardiac hypertrophy and/or heart failure comprising (a) providing a histone deacetylase inhibitor; (b) treating a myocyte with said histone deacetylase inhibitor; and (c) measuring the activity of at least HDAC1, HDAC2 and HDAC3, wherein a relative decrease in the activity of HDAC1 and/or HDAC2 versus HDAC3, as compared to an untreated myocyte, identifies said histone deacetylase inhibitor as an inhibitor of pathologic cardiac hypertrophy and/or heart failure. The myocyte may be subjected to a stimulus that triggers a hypertrophic response, such as expression of a transgene, or treatment with a drug. The hypertrophic response may comprise an alteration in the expression level of one or more target genes in said myocyte, wherein expression level of said one or more target genes is indicative of cardiac hypertrophy, such as one or more target genes is selected from the group consisting of ANF, α-MyHC, β-MyHC, α-skeletal actin, SERCA, cytochrome oxidase subunit VIII, mouse T-complex protein, insulin growth factor binding protein, Tau-microtubule-associated protein, ubiquitin carboxyl-terminal hydrolase, Thy-1 cell-surface glycoprotein, or MyHC class I antigen. Hypertrophic response may also comprise an alteration in one or more aspects of cellular morphology, such as sarcomere assembly, cell size, or cell contractility. The hypertrophic response may comprise an alteration in total protein synthesis. Activity may be assessed by measuring release of a labeled acetyl group from a histone. Alternatively, activity may be assessed by measuring the expression of (i) at least one of T-type Ca²⁺ channels, L-type Ca²⁺ channels, ssTnI, and fsTn1, and (ii) at least one of a myocardial energetic gene and/or a gene involved in glucose utilization. For example, measuring the expression may comprise measuring expression of a reporter protein, such as luciferase, β-gal, or green fluorescent protein, operably connected to a promoter for (i) at least one of T-type Ca²⁺ channels, L-type Ca²⁺ channels, ssTnI, and fsTn1, and (ii) at least one of a myocardial energetic gene and/or a gene involved in glucose utilization.

The myocyte may be an isolated myocyte, or comprised in isolated intact tissue. The myocyte may be a cardiomyocyte, such as a cardiomyocyte located in vivo in a functioning intact heart muscle. The functioning intact heart muscle may be subjected to a stimulus that triggers a hypertrophic response in said intact heart muscle, such as a pharmacologic stimulus, aortic banding, rapid cardiac pacing, induced myocardial infarction, or transgene expression. The hypertrophic response may comprise an alteration in right ventricle ejection fraction, left ventricle ejection fraction, ventricular wall thickness, heart weight/body weight ratio, and/or cardiac weight normalization measurement.

In still another embodiment, there is provided a transgenic non-human animal, cells of which lack at least one functional allele of HDAC3. The animal may lack two functional alleles of HDAC3. The animal may be a rat or a mouse.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-C. Generation of a conditional HDAC3 allele. (FIG. 1A) Strategy to generate a conditional HDAC3 allele. Protein, genomic structure, targeting vector, and targeting allele are shown. loxP sites were inserted upstream of exon 11 and downstream of exon 14. The neomycin cassette was removed by crossing to FLPe transgenic mice. Cre-mediated excision leaves one loxP site in place of exons 11 through 14. (FIG. 1B) Representative Southern blot of genomic DNA to show germline transmission. WT (˜13.8 kb) and targeted (˜6.6 kb) bands are indicated. (FIG. 1C) Genotyping of HDAC3 conditional mice by genomic PCR. Primer triplex includes one set flanking the 5′ loxP site and a third primer downstream of the 3′ loxP site. Global deletion by CAG-Cre removes the primer within the loxP sites, resulting in one ˜650-bp fragment. Cardiac deletion of HDAC3 results in deletion of exons 11 through 14. Primers are shown in (FIG. 1A).

FIGS. 2A-C. Cardiac-specific deletion of HDAC3. (FIG. 2A) Semi-quantitative RT-PCR showing HDAC3 transcript levels in wild-type and HDAC3cKO mice using primers in exon 10 forward and exon 15 reverse or exon 13 forward and 15 reverse. Cardiac deletion of HDAC3 results in deletion of exons 11 through 14. (FIG. 2B) Western blot showing reduced expression of HDAC3 in HDAC3cKO hearts. (FIG. 2C) Real-time PCR of class I and class II HDACs in wild-type and HDAC3cKO hearts.

FIGS. 3A-E. Cardiac defects resulting from cardiac deletion of HDAC3. (FIG. 3A) HW/BW ratios of WT and HDAC3cKO showing progression of cardiac hypertrophy. (FIG. 3B) Kaplan-Meier survival curve showing lethality by 16 weeks in HDAC3cKO mice. (FIG. 3C) Masson's trichrome stained sections of wild-type and HDAC3cKO mice at 12 weeks. Deletion of HDAC3 results in cardiac hypertrophy, left atrial thrombus, and cardiac fibrosis seen in blue. (FIG. 3D) Expression of cardiac stress markers in HDAC3cKO mice at 8 weeks. mRNA transcript levels were detected by real-time RT-PCR and normalized to 18S ribosomal RNA. (FIG. 3E) Electron microscopy of left ventricle tissue from WT and HDAC3cKO hearts at 8 weeks. Bar represents 5000 nm at 4200× and 1000 nm at 16500×. KO, HDAC3cKO.

FIG. 4. Echocardiographic data of HDAC3cKO mice. Values show severe ventricular dysfunction in HDAC3cKO hearts at 12 weeks of age. Values represent mean (+SEM). LVIDd, LV internal diameter at diastole; LVIDs, LV internal diameter at systole; FS, fractional shortening; KO, HDAC3cKO.

FIGS. 5A-E. Aberrant expression of cardiac metabolism genes from cardiac deletion of HDAC3. (FIG. 5A) Microarray analysis was performed at 5 weeks and gene ontology analysis was performed with PANTHER. Significantly enriched biological processes are shown and plotted as the −log(P value). (FIG. 5B) PPAR-regulated mitochondrial uncoupling genes are increased in HDAC3cKO mice. (FIG. 5C and FIG. 5D) Fatty acid uptake and oxidation genes are moderately increased in HDAC3cKO hearts. (FIG. 5E) Glucose metabolism is decreased in HDAC3cKO hearts. For (FIGS. 5B-E), real-time RT-PCR was performed from LV RNA of 6-week-old wild-type and HDAC3cKO mice in absence or presence of Wy14,643. Transcript levels were normalized to 18S Ribosomal RNA. KO, HDAC3cKO. Error bars represent standard deviation.

FIGS. 6A-C. Local promoter architecture of dysregulated transcripts. (FIG. 6A) ChIP assays were performed from neonatal rat myocytes. Chromatin was immunoprecipitated with antibodies against HA, HDAC3, or PPARα. Primers flank the PPAR-responsive elements of each gene and precipitated DNA was analyzed by PCR. Non-immunoprecipitated sample served as an input control. (FIG. 6B) Global histone acetylation is unchanged in HDAC3cKO hearts. Histones were isolated from wild-type and HDAC3cKO hearts and subjected to western blot analysis using antibodies against acetyl-H3, acetyl-H4, pan-acetyl-lysine, and H3. (FIG. 6C) Representative quantitative ChIP on dysregulated transcripts performed in triplicate from myocytes isolated from wild-type and HDAC3cKO hearts using anti-acetyl-H3 for immunoprecipitation. KO, HDAC3cKO.

FIGS. 7A-C. Myocardial lipid accumulation and mitochondrial dysfunction in HDAC3cKO mice. (FIG. 7A) Increased triglycerides in HDAC3cKO hearts after fasting. Triglycerides were extracted from wild-type and HDAC3cKO hearts at 8 weeks and quantified. (FIG. 7B) Oil red O staining of 8-week-old wild-type and cardiac deletion of HDAC3 following a 24 hr fast. Red droplets indicate neutral lipids. HDAC3cKO mice show substantial lipid accumulation. (FIG. 7C) HDAC3cKO hearts show impaired mitochondrial function. Complex I activity, NADH oxidase activity, and free radicals were determined from 8-week-old wild-type and HDAC3cKO mice. KO, HDAC3cKO.

### DESCRIPTION OF ILLUSTRATIVE EMBODIMENTS

Heart failure is one of the leading causes of morbidity and mortality in the world. In the U.S. alone, estimates indicate that 3 million people are currently living with cardiomyopathy and another 400,000 are diagnosed on a yearly basis. Dilated cardiomyopathy (DCM), also referred to as “congestive cardiomyopathy,” is the most common form of the cardiomyopathies and has an estimated prevalence of nearly 40 per 100,000 individuals (Durand et al., 1995). Although there are other causes of DCM, familiar dilated cardiomyopathy has been indicated as representing approximately 20% of “idiopathic” DCM. Approximately half of the DCM cases are idiopathic, with the remainder being associated with known disease processes. For example, serious myocardial damage can result from certain drugs used in cancer chemotherapy (e.g., doxorubicin and daunoribucin). In addition, many DCM patients are chronic alcoholics. Fortunately, for these patients, the progression of myocardial dysfunction may be stopped or reversed if alcohol consumption is reduced or stopped early in the course of disease. Peripartum cardiomyopathy is another idiopathic form of DCM, as is disease associated with infectious sequelae. In sum, cardiomyopathies, including DCM, are significant public health problems.

As cardiomyopathy itself typically does not produce any symptoms until the cardiac damage is severe enough to produce heart failure, the symptoms of cardiomyopathy are those associated with heart failure. These symptoms include shortness of breath, fatigue with exertion, the inability to lie flat without becoming short of breath (orthopnea), paroxysmal nocturnal dyspnea, enlarged cardiac dimensions, and/or swelling in the lower legs. Patients also often present with increased blood pressure, extra heart sounds, cardiac murmurs, pulmonary and systemic emboli, chest pain, pulmonary congestion, and palpitations. In addition, DCM causes decreased ejection fractions (i.e., a measure of both intrinsic systolic function and remodeling). The disease is further characterized by ventricular dilation and grossly impaired systolic function due to diminished myocardial contractility, which results in dilated heart failure in many patients. Affected hearts also undergo cell/chamber remodeling as a result of the myocyte/myocardial dysfunction, which contributes to the “DCM phenotype.” As the disease progresses, the symptoms progress as well. Patients with dilated cardiomyopathy also have a greatly increased incidence of life-threatening arrhythmias, including ventricular tachycardia and ventricular fibrillation. In these patients, an episode of syncope (dizziness) is regarded as a harbinger of sudden death.

Diagnosis of dilated cardiomyopathy typically depends upon the demonstration of enlarged heart chambers, particularly enlarged ventricles. Enlargement is commonly observable on chest X-rays, but is more accurately assessed using echocardiograms. DCM is often difficult to distinguish from acute myocarditis, valvular heart disease, coronary artery disease, and hypertensive heart disease. Once the diagnosis of dilated cardiomyopathy is made, every effort is made to identify and treat potentially reversible causes and prevent further heart damage. For example, coronary artery disease and valvular heart disease must be ruled out. Anemia, abnormal tachycardias, nutritional deficiencies, alcoholism, thyroid disease and/or other problems need to be addressed and controlled.

During attempts to identify and stabilize the underlying cause of the cardiomyopathy, treatment is generally instituted in order to minimize the symptoms and optimize the efficiency of the failing heart. Medication remains the mainstay of treatment, although there are no specific treatments for dilated cardiomyopathy other than those used in heart failure cases in general. Transplant surgery is one option. Indeed, dilated cardiomyopathy has been indicated as the most common cause for cardiac transplantation in the United States.

Non-pharmacological treatment is primarily used as an adjunct to pharmacological treatment. One means of non-pharmacological treatment involves reducing the sodium in the diet. In addition, non-pharmacological treatment also entails the elimination of certain precipitating drugs, including negative inotropic agents (e.g., certain calcium channel blockers and antiarrhythmic drugs like disopyramide), cardiotoxins (e.g., amphetamines), and plasma volume expanders (e.g., nonsteroidal anti-inflammatory agents and glucocorticoids).

Treatment with pharmacological agents represents the primary mechanism for reducing or eliminating the manifestations of heart failure. Diuretics constitute the first line of treatment for mild-to-moderate heart failure. Unfortunately, many of the commonly used diuretics (e.g., the thiazides) have numerous adverse effects. For example, certain diuretics may increase serum cholesterol and triglycerides. Moreover, diuretics are generally ineffective for patients suffering from severe heart failure.

If diuretics are ineffective, vasodilatory agents may be used; the angiotensin converting (ACE) inhibitors (e.g., enalopril and lisinopril) not only provide symptomatic relief, they also have been reported to decrease mortality (Young et al., 1989). Again, however, the ACE inhibitors are associated with adverse effects that result in their being contraindicated in patients with certain disease states (e.g., renal artery stenosis).

Similarly, inotropic agent therapy (i.e., a drug that improves cardiac output by increasing the force of myocardial muscle contraction) may also be indicated if the diuretics do not result in adequate relief. The inotropic agent most commonly used by ambulatory patients is digitalis. However, it is associated with a panoply of adverse reactions, including gastrointestinal problems and central nervous system dysfunction.

Thus, the currently used pharmacological agents have shortcomings, and the availability of new, safe and effective agents would undoubtedly benefit patients who either cannot use the pharmacological modalities presently available, or who do not receive adequate relief from those modalities.

The present inventors now reveal a unique and unexpected role of HDAC3 as a key regulator of cardiac energy metabolism. The inventors generated mice with a conditional null allele of HDAC3. While global deletion of HDAC3 results in embryonic lethality prior to E9.5, cardiac-specific deletion of HDAC3 results in cardiac hypertrophy and fibrosis by 3 months of age and lethality by 4 months. These mice show up-regulation of genes involved in fatty acid uptake and oxidation, down-regulation of the glucose utilization pathway, and ligand-induced myocardial lipid accumulation due to increased activity of the nuclear hormone receptor PPARα, a well known regulator of metabolism. Additionally, these hearts show mitochondrial dysfunction and decreased cardiac efficiency. The severe hypertrophy and metabolic abnormalities associated with deletion of HDAC3 contrast with the lack of a cardiac phenotype upon deletion of either HDAC1 or HDAC2 or the ventricular dilatation without metabolic abnormalities seen in mice with combined deletion of both HDAC1 and HDAC2 (Montgomery et al., 2007). Thus, despite the high degree of homology between HDAC1, HDAC2 and HDAC3, HDAC3 clearly plays a unique role in maintenance of cardiac function.

Maintenance of myocardial energy metabolism requires a precise balance of nuclear receptor activation and repression. The PPAR and estrogen-related receptor (ERR) families of nuclear hormone receptors control cardiac energetics through the activation of target genes to meet the metabolic demands of the heart during stress (Huss and Kelly, 2004). Under basal conditions, HDAC3, together with the NCoR/SMRT or the Rb complex, is specifically recruited by PPARs and other nuclear receptors to the promoters of target genes to facilitate the transcriptional repression by nuclear receptors (Guan et al., 2005; Fajas et al., 2002). PPARα and PPARδ function as central regulators of cardiac fatty acid metabolism (Finck et al., 2002; Kersten et al., 1999; Watanabe et al., 2000; Cheng et al., 2004). Consistent with the inventors' phenotype, cardiac-specific overexpression of PPARα causes increased expression of genes associated with mitochondrial uncoupling, fatty acid uptake and oxidation, and concomitant decreased expression of genes associated with glucose uptake, mimicking diabetic cardiomyopathy (Finck et al., 2002). Surprisingly, cardiac-specific overexpression of PPAR results in a metabolic phenotype independent from that of overexpression of PPARα, indicating separate PPAR isoforms control specific metabolic programs in the adult heart (Burkart et al., 2007). Furthermore, PPARδ overexpression does not result in cardiomyopathy or ligand-induced lipid accumulation, implying that the phenotype from loss of HDAC3 in cardiomyocytes is likely due to rampant PPARα activation and not other PPAR family members. Nevertheless, the inventors cannot rule out the additional activation of other nuclear receptors (such as ERRα) that depend upon HDAC3 and NCoR/SMRT to mediate repression. While ERRα and PPARα co-regulate multiple downstream target genes, ERRα has been shown to directly induce PPARα expression and PPARα is required for ERRα induction of β-oxidation genes (Huss et al., 2004). PPARα expression is unchanged in HDAC3cKO hearts suggesting the metabolic derangements in HDAC3cKO hearts primarily arise from PPARα activity and not ERRα; however, the inventors are currently investigating other ERRα-dependent mechanisms in HDAC3cKO mice. Additionally, HDAC3 has recently been shown to regulate cholesterol synthesis independent of nuclear receptors, such as through the recruitment of YY1 (Villagra et al., 2007). Current experiments are underway to identify additional transcription factors contributing to the metabolic derangements from loss of HDAC3 in cardiomyocytes.

The mitochondrial derangements from loss of HDAC3 in cardiomyocytes also resemble those seen in diabetic cardiomyopathies. The increased oxidation of fatty acids in diabetic hearts results in augmented reducing equivalents to the electron transport chain resulting in free radical production, mitochondrial uncoupling, and decreased cardiac efficiency (Boudina and Abel, 2006). The increase in UCP2 and UCP3 expression suggests mitochondria of HDAC3cKO mice are uncoupled and the increased free radical production and defects in electron transport are potential contributors to the ventricular dysfunction. Given the increase in expression of genes involved in oxidative phosphorylation and electron transport, as revealed by microarray analysis, these findings suggest either HDAC3 directly regulates these genes through ERRα or additional mechanisms, or a feedback mechanism exists that activates these genes to compensate for the electron transport chain inhibition and reduction in cardiac efficiency. Generation of reactive lipid intermediates from mitochondrial dysfunction in diabetic cardiomyopathies has been proposed as a “lipotoxic” mechanism of lipid-induced myocyte death leading to cardiac dysfunction. Surprisingly, no difference in apoptosis was observed between HDAC3cKO and wild-type mice by TUNEL analysis (data not shown), however, this does not rule out additional mechanisms of cell loss that are currently being investigated.

The abnormalities in cardiac metabolism resulting from loss of HDAC3 in cardiomyocytes contrasts with the phenotypes resulting from genetic deletion of other class I HDACs. Deletion of HDAC1 or HDAC2 individually in cardiomyocytes causes no phenotypic abnormalities, however deletion of HDAC1 and HDAC2 together in cardiomyocytes results in lethality by 2 weeks of age, accompanied by arrhythmias, dilated cardiomyopathy, and misexpression of calcium channels and contractile proteins (Montgomery et al., 2007). Mice lacking HDAC1 and HDAC2 also show no metabolic abnormalities, further illustrating the distinct functions of HDAC3 versus the functionally redundant HDAC1 and HDAC2 enzymes in cardiac growth and development. HDAC3 has classically been considered distinct from HDAC1 and HDAC2 in transcriptionally repressive complexes, associating with NCoR/SMRT as opposed to the CoREST, Sin3, and NuRD complexes that contain HDAC1 and HDAC2 (Grozinger and Schreiber, 2002). The results of this study further support differing repressive complexes in vivo for class I HDACs.

Other studies have indirectly implicated HDAC3 in the control of cardiac growth. Class II HDACs act as anti-hypertrophic mediators through the binding and repression of MEF2 and other transcription factors in the adult heart, however class II HDACs do not possess intrinsic deacetylase activity but instead, recruit the N-CoR/SMRT-HDAC3 complex to mediate deacetylation and transcriptional repression (Fischle et al., 2002). Additionally, HDAC3 has been shown to directly bind and deacetylate MEF2, thereby regulating MEF2 activity (Gregoire et al., 2007). Cardiac-specific overexpression of MEF2 results in cardiomyopathy with extensive fibrosis (Kim et al., 2008), however MEF2 activity was only slightly increased in hearts of HDAC3cKO mice as assayed by crossing HDAC3cKO mice to reporter mice harboring a MEF2-dependent transgene. These results indicate the metabolic derangements and cardiomyopathy associated with loss of HDAC3 are unlikely to be due to excessive MEF2 activity, but rather to rampant nuclear receptor-dependent activation. It should also be pointed out that HDAC3cKO mice do not phenocopy all aspects of diabetic cardiomyopathy. The excessive fibrosis seen in HDAC3cKO hearts, for example, contrasts with previous mouse models of diabetic cardiomyopathy. Multiple collagens and extracellular matrix proteins are significantly up-regulated in the HDAC3cKO hearts, however it is currently unclear whether these are direct targets of HDAC3 or, alternatively, if they are up-regulated as a secondary consequence of cardiac dysfunction.

The distinct phenotypes associated with loss of function studies from class I HDACs in cardiac hypertrophy underscore the necessity for more thorough analyses of specific roles of individual class I HDACs in cardiac physiology and pathology. Deletion of HDAC1 or HDAC2 individually in cardiomyocytes did not affect the response to hypertrophic stimuli compared to wild-type littermates, however mice from a gene-trap deletion of HDAC2 fail to undergo cardiac hypertrophy during aortic constriction or β-adrenergic stimulation (Montgomery et al., 2007; Trivedi et al., 2007), potentially pointing to a role for histone deacetylases in cardiac fibroblasts as a mechanism for regulating the hypertrophic response. Conversely, deletion of HDAC3 in cardiomyocytes resulted in robust cardiac hypertrophy from metabolic derangements. Furthermore, cardiac-specific overexpression of HDAC2 induces significant hypertrophy by 8 weeks of age (Trivedi et al., 2007), whereas αMHC-HDAC3 transgenic mice showed no basal phenotype, but did exhibit stress-dependent cardiomegaly (data not shown). Additionally, the inventors generated cardiac-specific transgenics for HDAC1 and HDAC2; and these mice showed robust cardiac hypertrophy, cardiac dilatation, and sudden death dependent upon the level of overexpression (data not shown). Together, these studies illustrate the contrasting roles for HDAC1/HDAC2 and HDAC3 in cardiomyopathies and show class I HDACs serve as distinct nodal points in the precise regulation of gene expression to maintain cardiac function.

Class I HDAC inhibitors have shown benefit in a variety of disease states and the first HDAC inhibitor has just been granted approval by the FDA for cutaneous T-cell lymphoma. Recent studies have implicated class I HDACs as the primary target of broad-spectrum HDAC inhibitors in vivo, however the specificity of HDAC inhibitors for class I HDACs has remained enigmatic. Current class I HDAC inhibitors do not show significant specificity for HDAC1 and HDAC2 over HDAC3. Given the widespread efforts to develop HDAC inhibitors for numerous disorders, the phenotype of HDAC3 mutant mice emphasizes the need to avoid HDAC3 inhibition so as to avoid cardiac toxicity. As such, the present invention provides for a more selective version of HDAC inhibition, namely, inhibition of HDACS1 and 2 without inhibition of substantial HDAC3. These and other aspects of the invention are provided below.

### I. HISTONE DEACETYLASES

Nucleosomes, the primary scaffold of chromatin folding, are dynamic macromolecular structures, influencing chromatin solution conformations (Workman and Kingston, 1998). The nucleosome core is made up of histone proteins, H2A, HB, H3 and H4. Histone acetylation causes nucleosomes and nucleosomal arrangements to behave with altered biophysical properties. The balance between activities of histone acetyl transferases (HAT) and deacetylases (HDAC) determines the level of histone acetylation. Acetylated histones cause relaxation of chromatin and activation of gene transcription, whereas deacetylated chromatin generally is transcriptionally inactive.

Eleven different HDACs have been cloned from vertebrate organisms. The first three human HDACs identified were HDAC 1, HDAC 2 and HDAC 3 (termed class I human HDACs), and HDAC 8 (Van den Wyngaert et al., 2000) has been added to this list. Class II human HDACs, HDAC 4, HDAC 5, HDAC 6, HDAC 7, HDAC 9, and HDAC 10 (Kao et al., 2000) have been cloned and identified (Grozinger et al., 1999; Zhou et al. 2001; Tong et al., 2002). Class III HDACs are the sirtuins and homologs thereof. Additionally, HDAC 11 was later identified and is now classified as class IV.

HDACs 4, 5, 7, 9 and 10 have a unique amino-terminal extension not found in other HDACs. This amino-terminal region contains the MEF2-binding domain. HDACs 4, 5 and 7 have been shown to be involved in the regulation of cardiac gene expression and in particular embodiments, repressing MEF2 transcriptional activity. The exact mechanism in which class II HDAC's repress MEF2 activity is not completely understood. One possibility is that HDAC binding to MEF2 inhibits MEF2 transcriptional activity, either competitively or by destabilizing the native, transcriptionally active MEF2 conformation. It also is possible that class II HDAC's require dimerization with MEF2 to localize or position HDAC in a proximity to histones for deacetylation to proceed.

A. Class II HDACs and Cardiac Growth

MEF2 is among a number of transcription factors whose expression remains constant during hypertrophy, but MEF2 activity is greatly increased. This is due to transcriptional regulation by the class IIa HDACs (HDAC4, HDAC5, HDAC7, and HDAC9). These proteins contain an amino-terminal extension that mediates interaction with various transcription factors. Mice with genetic deletion of HDAC5 or HDAC9 are viable; however, these mice begin to show cardiac abnormalities at about 6 months of age. Additionally, these mice are hypersensitive to certain calcium-dependent stresses such thoracic aortic constriction, showing an exacerbated hypertrophic response. The early lethality associated with loss of HDAC4 or HDAC7 has precluded the analysis of these proteins in modulating the hypertrophic program.

The exacerbation of the hypertrophic response in loss of HDAC5 or HDAC9 is presumably due to increased activity of the transcription factor MEF2. Under normal physiological conditions, class IIa HDACs bind to MEF2 and block MEF2-dependent gene activation. Extrinsic stress signals are able to activate various intracellular kinases such as CaMKII and PKD that directly phosphorylate class IIa HDACs at 2 conserved serine residues. This phosphorylation results in the binding of the chaperone 14-3-3, nucleocytoplasmic shuttling by CRM1, and derepression of MEF2 and other transcription factors. The loss of HDAC5 or HDAC9 results in an imbalance of MEF2 activity within the myocytes and subsequent activation of the hypertrophic response.

B. HDAC Inhibitors and Class I HDACs in Hypertrophy

Given the loss of function phenotypes of HDAC5 or HDAC9, one might predict treatment of cardiomyocytes with HDAC inhibitors would result in cardiac hypertrophy. Paradoxically, HDAC inhibitors such as TSA and VPA are able to block the hypertrophic response and dose-dependentle block activation of the fetal gene program. Surviving mice from a gene-trap deletion of HDAC2 block cardiac hypertrophy from aortic constriction and β-adrenergic stimulation, lending support for HDAC2 as a nodal point in the activation of the fetal gene program during hypertrophy. The inventors have shown that HDAC1 and HDAC2 act redundantly in the pathological setting of hypertrophy as cardiac-specific deletion of HDAC1 or HDAC2 is not able to block the hypertrophic response. Cardiac-specific deletion of both HDAC1 and HDAC2 results in neonatal lethality, precluding adult hypertrophy analysis. These studies have implicated class I HDACs as critical regulators of the hypertrophic response. Interestingly, these enzymes appear to play opposing roles to class II HDACs during pathological hypertrophy. Because of this, the design of more specific inhibitors against class I HDACs remains optimistic for the treatment of cardiac hypertrophy and heart failure.

### II. DEACETYLASE INHIBITORS

The present invention provides, for the first time, an understanding of the differential functions, and hence differential effects from inhibition, of class I HDACs. In particular, cardiac-specific knockout of HDAC 1 or 2 in mice does not result in an apparent phenotype. In contrast, cardiac-specific knockout of HDAC3 resulted in massive hypertrophy at 3 to 4 months of age, indicating that global repression of Class I HDACs can have negative consequences for treatment of pathologic hypertrophy.

Therefore, the present inventors propose selective, indeed highly selective, inhibition of HDAC1 and/or 2 over HDAC3. It also may prove useful to avoid inhibition of HDAC8 in addition to HDAC3. By selective, it is meant that inhibition of HDAC1 or HDAC2 should be greater than inhibition of HDAC3 by a given agent or therapy. The inhibition maybe 2-, 3-, 4-, 5-, 6-, 7-, 8-, 9-, 10-, 15-, 20-, 25-, 30-, 40-, 50-, 60-, 70-, 80-, 90-, 100-, 150-, 200-, 250-, 500-, 1000-, 2500-, 5000-, 10,000-, 25,000-, 50,000-fold or greater.

Of particular interest in the present invention are various small molecule drugs that can selectively inhibit HDACs 1 and 2 over 3. A series of compounds have been described that can achieve selective inhibition—these are generally referred to as biaryl (e.g., phenyl and thiophenyl) benzamides (Witter et al., 2008; Methot et al., 2008). Another related class of HDAC1/2 selective inhibitors are the aminophenyl benzamides, and in particular, para-substituted derivatives thereof (Moradei et al., 2007). Such compounds may be exemplified by the following:

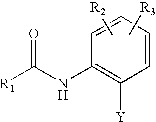

wherein:
   Y is amino or hydroxy;
   R₁ is aryl_{(C6-18)} or heteroaryl_{(C5-17)}, or a substituted version of either group;
   R₂ and R₃ are independently:
      hydrogen, hydroxy, halo, amino, hydroxyamino, mercapto; or
      alkyl_{(C≦12)}, alkenyl_{(C≦12)}, alkynyl_{(C≦12)}, aryl_{(C≦12)}, aralkyl_{(C≦12)}, heteroaryl_{(C≦12)}, heteroaralkyl_{(C≦12)}, alkoxy_{(C≦12)}, alkenyloxy_{(C≦12)}, alkynyloxy_{(C≦12)}, aryloxy_{(C≦12)}, aralkoxy_{(C≦12)}, heteroaryloxy_{(C≦12)}, heteroaralkoxy_{(C≦12)}, acyloxy_{(C≦12)}, alkylamino_{(C≦12)}, dialkylamino_{(C≦12)}, alkoxyamino_{(C≦12)}, alkenylamino_{(C≦12)}, alkynylamino_{(C≦12)}, arylamino_{(C≦12)}, aralkylamino_{(C≦12)}, heteroarylamino_{(C≦12)}, heteroaralkylamino_{(C≦12)}, alkylsulfonylamino_{(C≦12)}, amido_{(C≦12)}, alkylthio_{(C≦12)}, arylthio_{(C≦12)}, aralkylthio_{(C≦12)}, heteroarylthio_{(C≦12)}, heteroaralkylthio_{(C≦12)}, alkylammonium_{(C≦12)}, alkylsilyl_{(C≦12)}, or a substituted version of any of these groups;
or pharmaceutically acceptable salts, hydrates, solvates, tautomers, prodrugs, or optical isomers thereof.

HDACs can also be inhibited through the use of biologicals—proteins (including antibodies), peptides, nucleic acids (including antisense and RNAi molecules), and small molecules. Methods are widely known to those of skill in the art for the cloning, transfer and expression of gene products, which include viral and non-viral vectors, and liposomes for delivery of both nucleic acids and proteins. Viral vectors include adenovirus, adeno-associated virus, retrovirus, vaccina virus and herpesvirus.

### III. METHODS OF TREATING CARDIAC HYPERTROPHY

### A. Therapeutic Regimens

In one embodiment of the present invention, methods for the treatment of cardiac hypertrophy utilizing HDAC1/2 selective inhibitors are provided. For the purposes of the present application, treatment comprises reducing one or more of the symptoms of cardiac hypertrophy, such as reduced exercise capacity, reduced blood ejection volume, increased left ventricular end diastolic pressure, increased pulmonary capillary wedge pressure, reduced cardiac output, cardiac index, increased pulmonary artery pressures, increased left ventricular end systolic and diastolic dimensions, and increased left ventricular wall stress, wall tension and wall thickness-same for right ventricle. In addition, use of HDAC1/2 selective inhibitors may prevent cardiac hypertrophy and its associated symptoms from arising.

Treatment regimens would vary depending on the clinical situation. However, long term maintenance would appear to be appropriate in most circumstances. It also may be desirable treat hypertrophy with HDAC1/2 selective inhibitors intermittently, such as within brief window during disease progression. At present, testing indicates that the optimal dosage for an HDAC1/2 selective inhibitor will be the maximal dose before significant toxicity occurs.

### B. Combined Therapy

In another embodiment, it is envisioned to use HDAC1/2 selective inhibition in combination with other therapeutic modalities. Thus, in addition to the therapies described above, one may also provide to the patient more “standard” pharmaceutical cardiac therapies. Examples of standard therapies include, without limitation, so-called “beta blockers,” anti-hypertensives, cardiotonics, anti-thrombotics, vasodilators, hormone antagonists, iontropes, diuretics, endothelin antagonists, calcium channel blockers, phosphodiesterase inhibitors, ACE inhibitors, angiotensin type 2 antagonists and cytokine blockers/inhibitors.

Combinations may be achieved by contacting cardiac cells with a single composition or pharmacological formulation that includes both agents, or by contacting the cell with two distinct compositions or formulations, at the same time, wherein one composition includes the expression construct and the other includes the agent. Alternatively, the HDAC1/2 selective inhibitor therapy may precede or follow administration of the other agent by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either an HDAC1/2 selective inhibitor, or the other agent will be desired. In this regard, various combinations may be employed. By way of illustration, where the HDAC1/2 selective inhibitor is “A” and the other agent is “B,” the following permutations based on 3 and 4 total administrations are exemplary:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are likewise contemplated. The following treatments are contemplated for combination with HDAC1/2 selective inhibitors.

i. Antihyperlipoproteinemics

In certain embodiments, administration of an agent that lowers the concentration of one of more blood lipids and/or lipoproteins, known herein as an “antihyperlipoproteinemic,” may be combined with a cardiovascular therapy according to the present invention, particularly in treatment of athersclerosis and thickenings or blockages of vascular tissues. In certain aspects, an antihyperlipoproteinemic agent may comprise an aryloxyalkanoic/fibric acid derivative, a resin/bile acid sequesterant, a HMG CoA reductase inhibitor, a nicotinic acid derivative, a thyroid hormone or thyroid hormone analog, a miscellaneous agent or a combination thereof.

a. Aryloxyalkanoic Acid/Fibric Acid Derivatives

Non-limiting examples of aryloxyalkanoic/fibric acid derivatives include beclobrate, enzafibrate, binifibrate, ciprofibrate, clinofibrate, clofibrate (atromide-S), clofibric acid, etofibrate, fenofibrate, gemfibrozil (lobid), nicofibrate, pirifibrate, ronifibrate, simfibrate and theofibrate.

b. Resins/Bile Acid Sequesterants

Non-limiting examples of resins/bile acid sequesterants include cholestyramine (cholybar, questran), colestipol (colestid) and polidexide.

c. HMG CoA Reductase Inhibitors

Non-limiting examples of HMG CoA reductase inhibitors include lovastatin (mevacor), pravastatin (pravochol) or simvastatin (zocor).

d. Nicotinic Acid Derivatives

Non-limiting examples of nicotinic acid derivatives include nicotinate, acepimox, niceritrol, nicoclonate, nicomol and oxiniacic acid.

e. Thyroid Hormones and Analogs

Non-limiting examples of thyroid hormones and analogs thereof include etoroxate, thyropropic acid and thyroxine.

f. Miscellaneous Antihyperlipoproteinemics

Non-limiting examples of miscellaneous antihyperlipoproteinemics include acifran, azacosterol, benfluorex, β-benzalbutyramide, carnitine, chondroitin sulfate, clomestrone, detaxtran, dextran sulfate sodium, 5,8,11,14,17-eicosapentaenoic acid, eritadenine, furazabol, meglutol, melinamide, mytatrienediol, ornithine, γ-oryzanol, pantethine, pentaerythritol tetraacetate, α-phenylbutyramide, pirozadil, probucol (lorelco), β-sitosterol, sultosilic acid-piperazine salt, tiadenol, triparanol and xenbucin.

ii. Antiarteriosclerotics

Non-limiting examples of an antiarteriosclerotic include pyridinol carbamate.

iii. Antithrombotic/Fibrinolytic Agents

In certain embodiments, administration of an agent that aids in the removal or prevention of blood clots may be combined with administration of a modulator, particularly in treatment of athersclerosis and vasculature (e.g., arterial) blockages. Non-limiting examples of antithrombotic and/or fibrinolytic agents include anticoagulants, anticoagulant antagonists, antiplatelet agents, thrombolytic agents, thrombolytic agent antagonists or combinations thereof.

In certain aspects, antithrombotic agents that can be administered orally, such as, for example, aspirin and wafarin (coumadin), are preferred.

a. Anticoagulants

A non-limiting example of an anticoagulant include acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin.

b. Antiplatelet Agents

Non-limiting examples of antiplatelet agents include aspirin, a dextran, dipyridamole (persantin), heparin, sulfinpyranone (anturane) and ticlopidine (ticlid).

c. Thrombolytic Agents

Non-limiting examples of thrombolytic agents include tissue plaminogen activator (activase), plasmin, pro-urokinase, urokinase (abbokinase) streptokinase (streptase), anistreplase/APSAC (eminase).

iv. Blood Coagulants

In certain embodiments wherein a patient is suffering from a hemmorage or an increased likelihood of hemmoraging, an agent that may enhance blood coagulation may be used. Non-limiting examples of a blood coagulation promoting agent include thrombolytic agent antagonists and anticoagulant antagonists.

a. Anticoagulant Antagonists

Non-limiting examples of anticoagulant antagonists include protamine and vitamin K1.

b. Thrombolytic Agent Antagonists and Antithrombotics

Non-limiting examples of thrombolytic agent antagonists include aminocaproic acid (amicar) and tranexamic acid (amstat). Non-limiting examples of antithrombotics include anagrelide, argatroban, cilstazol, daltroban, defibrotide, enoxaparin, fraxiparine, indobufen, lamoparan, ozagrel, picotamide, plafibride, tedelparin, ticlopidine and triflusal.

v. Antiarrhythmic Agents

Non-limiting examples of antiarrhythmic agents include Class I antiarrythmic agents (sodium channel blockers), Class II antiarrythmic agents (β-adrenergic blockers), Class II antiarrythmic agents (repolarization prolonging drugs), Class IV antiarrhythmic agents (calcium channel blockers) and miscellaneous antiarrythmic agents.

a. Sodium Channel Blockers

Non-limiting examples of sodium channel blockers include Class IA, Class IB and Class IC antiarrhythmic agents. Non-limiting examples of Class IA antiarrhythmic agents include disppyramide (norpace), procainamide (pronestyl) and quinidine (quinidex). Non-limiting examples of Class TB antiarrhythmic agents include lidocaine (xylocaine), tocamide (tonocard) and mexiletine (mexitil). Non-limiting examples of Class IC antiarrhythmic agents include encamide (enkaid) and flecamide (tambocor).

b. β Blockers

Non-limiting examples of a β blocker, otherwise known as a β-adrenergic blocker, a β-adrenergic antagonist or a Class II antiarrhythmic agent, include acebutolol (sectral), alprenolol, amosulalol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol (inderal), sotalol (betapace), sulfinalol, talinolol, tertatolol, timolol, toliprolol and xibinolol. In certain aspects, the beta blocker comprises an aryloxypropanolamine derivative. Non-limiting examples of aryloxypropanolamine derivatives include acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, moprolol, nadolol, nipradilol, oxprenolol, penbutolol, pindolol, propanolol, talinolol, tertatolol, timolol and toliprolol.

c. Repolarization Prolonging Agents

Non-limiting examples of an agent that prolong repolarization, also known as a Class III antiarrhythmic agent, include amiodarone (cordarone) and sotalol (betapace).

d. Calcium Channel Blockers/Antagonist

Non-limiting examples of a calcium channel blocker, otherwise known as a Class IV antiarrythmic agent, include an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a micellaneous calcium channel blocker such as bencyclane, etafenone, magnesium, mibefradil or perhexyline. In certain embodiments a calcium channel blocker comprises a long-acting dihydropyridine (nifedipine-type) calcium antagonist.

e. Miscellaneous Antiarrhythmic Agents

Non-limiting examples of miscellaneous antiarrhymic agents include adenosine (adenocard), digoxin (lanoxin), acecamide, ajmaline, amoproxan, aprindine, bretylium tosylate, bunaftine, butobendine, capobenic acid, cifenline, disopyranide, hydroquinidine, indecamide, ipatropium bromide, lidocaine, lorajmine, lorcamide, meobentine, moricizine, pirmenol, prajmaline, propafenone, pyrinoline, quinidine polygalacturonate, quinidine sulfate and viquidil.

vi. Antihypertensive Agents

Non-limiting examples of antihypertensive agents include sympatholytic, alpha/beta blockers, alpha blockers, anti-angiotensin II agents, beta blockers, calcium channel blockers, vasodilators and miscellaneous antihypertensives.

a. α Blockers

Non-limiting examples of an α blocker, also known as an α-adrenergic blocker or an α-adrenergic antagonist, include amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosin, doxazosin, prazosin, terazosin and trimazosin.

b. α/β Blockers

In certain embodiments, an antihypertensive agent is both an a and p adrenergic antagonist. Non-limiting examples of an α/β blocker comprise labetalol (normodyne, trandate).

c. Anti-Angiotension II Agents

Non-limiting examples of anti-angiotensin II agents include angiotensin converting enzyme inhibitors and angiotensin II receptor antagonists. Non-limiting examples of angiotensin converting enzyme inhibitors (ACE inhibitors) include alacepril, enalapril (vasotec), captopril, cilazapril, delapril, enalaprilat, fosinopril, lisinopril, moveltopril, perindopril, quinapril and ramipril. Non-limiting examples of an angiotensin II receptor blocker, also known as an angiotensin II receptor antagonist, an ANG receptor blocker or an ANG-II type-1 receptor blocker (ARBS), include angiocandesartan, eprosartan, irbesartan, losartan and valsartan.

d. Sympatholytics

Non-limiting examples of a sympatholytic include a centrally acting sympatholytic or a peripherially acting sympatholytic. Non-limiting examples of a centrally acting sympatholytic, also known as an central nervous system (CNS) sympatholytic, include clonidine (catapres), guanabenz (wytensin) guanfacine (tenex) and methyldopa (aldomet). Non-limiting examples of a peripherally acting sympatholytic include a ganglion blocking agent, an adrenergic neuron blocking agent, a β-adrenergic blocking agent or a α1-adrenergic blocking agent. Non-limiting examples of a ganglion blocking agent include mecamylamine (inversine) and trimethaphan (arfonad). Non-limiting of an adrenergic neuron blocking agent include guanethidine (ismelin) and reserpine (serpasil). Non-limiting examples of a β-adrenergic blocker include acenitolol (sectral), atenolol (tenormin), betaxolol (kerlone), carteolol (cartrol), labetalol (normodyne, trandate), metoprolol (lopressor), nadanol (corgard), penbutolol (levatol), pindolol (visken), propranolol (inderal) and timolol (blocadren). Non-limiting examples of α1-adrenergic blocker include prazosin (minipress), doxazocin (cardura) and terazosin (hytrin).

e. Vasodilators

In certain embodiments a cardiovasculator therapeutic agent may comprise a vasodilator (e.g., a cerebral vasodilator, a coronary vasodilator or a peripheral vasodilator). In certain preferred embodiments, a vasodilator comprises a coronary vasodilator. Non-limiting examples of a coronary vasodilator include amotriphene, bendazol, benfurodil hemisuccinate, benziodarone, chloracizine, chromonar, clobenfurol, clonitrate, dilazep, dipyridamole, droprenilamine, efloxate, erythrityl tetranitrane, etafenone, fendiline, floredil, ganglefene, herestrol bis(β-diethylaminoethyl ether), hexobendine, itramin tosylate, khellin, lidoflanine, mannitol hexanitrane, medibazine, nicorglycerin, pentaerythritol tetranitrate, pentrinitrol, perhexyline, pimethylline, trapidil, tricromyl, trimetazidine, trolnitrate phosphate and visnadine.

In certain aspects, a vasodilator may comprise a chronic therapy vasodilator or a hypertensive emergency vasodilator. Non-limiting examples of a chronic therapy vasodilator include hydralazine (apresoline) and minoxidil (loniten). Non-limiting examples of a hypertensive emergency vasodilator include nitroprusside (nipride), diazoxide (hyperstat IV), hydralazine (apresoline), minoxidil (loniten) and verapamil.

f. Miscellaneous Antihypertensives

Non-limiting examples of miscellaneous antihypertensives include ajmaline, γ-aminobutyric acid, bufeniode, cicletainine, ciclosidomine, a cryptenamine tannate, fenoldopam, flosequinan, ketanserin, mebutamate, mecamylamine, methyldopa, methyl 4-pyridyl ketone thiosemicarbazone, muzolimine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil.

In certain aspects, an antihypertensive may comprise an arylethanolamine derivative, a benzothiadiazine derivative, a N-carboxyalkyl(peptide/lactam) derivative, a dihydropyridine derivative, a guanidine derivative, a hydrazines/phthalazine, an imidazole derivative, a quanternary ammonium compound, a reserpine derivative or a suflonamide derivative.

Arylethanolamine Derivatives. Non-limiting examples of arylethanolamine derivatives include amosulalol, bufuralol, dilevalol, labetalol, pronethalol, sotalol and sulfinalol.

Benzothiadiazine Derivatives. Non-limiting examples of benzothiadiazine derivatives include althizide, bendroflumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydroflumethizide, methyclothiazide, meticrane, metolazone, paraflutizide, polythizide, tetrachlormethiazide and trichlormethiazide.

N-carboxyalkyl(peptide/lactam) Derivatives. Non-limiting examples of N-carboxyalkyl(peptide/lactam) derivatives include alacepril, captopril, cilazapril, delapril, enalapril, enalaprilat, fosinopril, lisinopril, moveltipril, perindopril, quinapril and ramipril.

Dihydropyridine Derivatives. Non-limiting examples of dihydropyridine derivatives include amlodipine, felodipine, isradipine, nicardipine, nifedipine, nilvadipine, nisoldipine and nitrendipine.

Guanidine Derivatives. Non-limiting examples of guanidine derivatives include bethanidine, debrisoquin, guanabenz, guanacline, guanadrel, guanazodine, guanethidine, guanfacine, guanochlor, guanoxabenz and guanoxan.

Hydrazines/Phthalazines. Non-limiting examples of hydrazines/phthalazines include budralazine, cadralazine, dihydralazine, endralazine, hydracarbazine, hydralazine, pheniprazine, pildralazine and todralazine.

Imidazole Derivatives. Non-limiting examples of imidazole derivatives include clonidine, lofexidine, phentolamine, tiamenidine and tolonidine.

Quanternary Ammonium Compounds. Non-limiting examples of quanternary ammonium compounds include azamethonium bromide, chlorisondamine chloride, hexamethonium, pentacynium bis(methylsulfate), pentamethonium bromide, pentolinium tartrate, phenactropinium chloride and trimethidinium methosulfate.

Reserpine Derivatives. Non-limiting examples of reserpine derivatives include bietaserpine, deserpidine, rescinnamine, reserpine and syrosingopine.

Suflonamide Derivatives. Non-limiting examples of sulfonamide derivatives include ambuside, clopamide, furosemide, indapamide, quinethazone, tripamide and xipamide.

g. Vasopressors

Vasopressors generally are used to increase blood pressure during shock, which may occur during a surgical procedure. Non-limiting examples of a vasopressor, also known as an antihypotensive, include amezinium methyl sulfate, angiotensin amide, dimetofrine, dopamine, etifelmin, etilefrin, gepefrine, metaraminol, midodrine, norepinephrine, pholedrine and synephrine.

vii. Treatment Agents for Congestive Heart Failure

Non-limiting examples of agents for the treatment of congestive heart failure include anti-angiotension II agents, afterload-preload reduction treatment, diuretics and inotropic agents.

a. Afterload-Preload Reduction

In certain embodiments, an animal patient that can not tolerate an angiotension antagonist may be treated with a combination therapy. Such therapy may combine administration of hydralazine (apresoline) and isosorbide dinitrate (isordil, sorbitrate).

b. Diuretics

Non-limiting examples of a diuretic include a thiazide or benzothiadiazine derivative (e.g., althiazide, bendroflumethazide, benzthiazide, benzylhydrochlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamphamide, mercaptomerin sodium, mercumallylic acid, mercumatilin dodium, mercurous chloride, mersalyl), a pteridine (e.g., furtherene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenamide, clofenamide, clopamide, clorexolone, diphenylmethane-4,4′-disulfonamide, disulfamide, ethoxzolamide, furosemide, indapamide, mefruside, methazolamide, piretanide, quinethazone, torasemide, tripamide, xipamide), a uracil (e.g., aminometradine, amisometradine), a potassium sparing antagonist (e.g., amiloride, triamterene) or a miscellaneous diuretic such as aminozine, arbutin, chlorazanil, ethacrynic acid, etozolin, hydracarbazine, isosorbide, mannitol, metochalcone, muzolimine, perhexyline, ticrnafen and urea.

c. Inotropic Agents

Non-limiting examples of a positive inotropic agent, also known as a cardiotonic, include acefylline, an acetyldigitoxin, 2-amino-4-picoline, aminone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, enoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol.

In particular aspects, an intropic agent is a cardiac glycoside, a beta-adrenergic agonist or a phosphodiesterase inhibitor. Non-limiting examples of a cardiac glycoside includes digoxin (lanoxin) and digitoxin (crystodigin). Non-limiting examples of a β-adrenergic agonist include albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Non-limiting examples of a phosphodiesterase inhibitor include aminone (inocor).

d. Antianginal Agents

Antianginal agents may comprise organonitrates, calcium channel blockers, beta blockers and combinations thereof.

Non-limiting examples of organonitrates, also known as nitrovasodilators, include nitroglycerin (nitro-bid, nitrostat), isosorbide dinitrate (isordil, sorbitrate) and amyl nitrate (aspirol, vaporole).

viii. Surgical Therapeutic Agents

In certain aspects, the secondary therapeutic agent may comprise a surgery of some type, which includes, for example, preventative, diagnostic or staging, curative and palliative surgery. Surgery, and in particular a curative surgery, may be used in conjunction with other therapies, such as the present invention and one or more other agents.

Such surgical therapeutic agents for vascular and cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

### C. Drug Formulations and Routes for Administration to Patients

Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target heart muscle cells. Aqueous compositions of the present invention comprise an effective amount of the agent(s), dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the vectors or cells of the compositions.

The active compositions of the present invention may include classic pharmaceutical preparations. Administration of these compositions according to the present invention may be via any common route so long as the target tissue is available via that route. This includes oral, sublingual, nasal, or buccal. Alternatively, administration may be by intradermal, subcutaneous, intramuscular, intraperitoneal or intravenous injection. Such compositions would normally be administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

For oral administration the polypeptides of the present invention generally may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including gels, pastes, powders and slurries. The active ingredient may be added in a therapeutically effective amount to a paste dentifrice that may include water, binders, abrasives, flavoring agents, foaming agents, and humectants.

The compositions of the present invention generally may be formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### IV. SCREENING METHODS

The present invention further comprises methods for identifying selective inhibitors of HDAC 1 and HDAC2 that are useful in the prevention or reversal of cardiac hypertrophy and heart failure. These assays may comprise random screening of large libraries of candidate substances; alternatively, the assays may be used to focus on particular classes of compounds selected with an eye towards structural attributes that are believed to make them more likely to inhibit the function of certain HDACs (HDAC1-2) while avoiding inhibition of other HDACs (HDAC3, and optionally HDAC8).

To identify a selective HDAC1/2 inhibitor, one generally will determine the function of various HDACs in the presence and absence of the candidate substance. For example, a method generally comprises:
(a) providing a candidate modulator;
(b) contacting the candidate modulator with an HDAC;
(c) measuring HDAC activity; and
(d) comparing the activity in step (c) with the activity in the absence of the candidate modulator.
   Agents that inhibit HDAC1/2, but not HDAC3/8, are considered useful selective inhibitors. Assays also may be conducted in isolated cells or in organisms. Typically, HDAC activity is measured by providing a histone with a labeled acetyl group and measuring release of the label from the histone molecule.

It will, of course, be understood that all the screening methods of the present invention are useful in themselves notwithstanding the fact that effective candidates may not be found. The invention provides methods for screening for such candidates, not solely methods of finding them.

A. Modulators

As used herein the term “candidate substance” refers to any molecule that may selectively inhibit HDAC1/2 activity as compared to HDAC 3 activity. The candidate substance may be a protein or fragment thereof, a small molecule, or even a nucleic acid. It may prove to be the case that the most useful pharmacological compounds will be compounds that are structurally related to known selective HDAC inhibitors, listed elsewhere in this document. Using lead compounds to help develop improved compounds is known as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecular libraries that are believed to meet the basic criteria for useful drugs in an effort to “brute force” the identification of useful compounds. Screening of such libraries, including combinatorially-generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, inhibitory RNAs, and antibodies (including single chain antibodies), each of which would be specific for the target molecule. Such compounds are described in greater detail elsewhere in this document. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

B. In Vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

A technique for high throughput screening of compounds is described in WO 84/03564. Large numbers of small peptide test compounds are synthesized on a solid substrate, such as plastic pins or some other surface. Such peptides could be rapidly screening for their ability to bind and inhibit the active site of specific HDACs.

C. In Cyto Assays

The present invention also contemplates the screening of compounds for their ability to selectively modulate HDAC1/2 expression and/or activity in cells. Various cell lines can be utilized for such screening assays, including cells specifically engineered for this purpose.

D. In Vivo Assays

In vivo assays involve the use of various animal models of heart disease, including transgenic animals, that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for inhibitors may be conducted using an animal model derived from any of these species.

Treatment of animals with test compounds will involve the administration of the compound, in an appropriate form, to the animal. Administration will be by any route that could be utilized for clinical purposes. Determining the effectiveness of a compound in vivo may involve a variety of different criteria, including but not limited to improvement in one or more of the symptoms of cardiac hypertrophy, such as reduced exercise capacity, reduced blood ejection volume, increased left ventricular end diastolic pressure, increased pulmonary capillary wedge pressure, reduced cardiac output, cardiac index, increased pulmonary artery pressures, increased left ventricular end systolic and diastolic dimensions, and increased left ventricular wall stress, wall tension and wall thickness-same for right ventricle. Also, measuring toxicity and dose response can be performed in animals in a more meaningful fashion than in in vitro or in cyto assays.

### V. METHODS OF MAKING TRANSGENIC MICE

A particular embodiment of the present invention provides transgenic animals that lack one or both alleles of HDAC3. Transgenic animals lacking HDAC3 allele(s), recombinant cell lines derived from such animals, and transgenic embryos may be useful in a variety of contexts.

In a general aspect, a transgenic animal is produced by the integration of a given transgene into the genome in a manner that permits the expression of the transgene. Methods for producing transgenic animals are generally described by Wagner and Hoppe (U.S. Pat. No. 4,873,191; which is incorporated herein by reference), and Brinster et al., 1985; which is incorporated herein by reference in its entirety).

Typically, a gene flanked by genomic sequences is transferred by microinjection into a fertilized egg. The microinjected eggs are implanted into a host female, and the progeny are screened for the expression of the transgene. Transgenic animals may be produced from the fertilized eggs from a number of animals including, but not limited to reptiles, amphibians, birds, mammals, and fish.

DNA clones for microinjection can be prepared by any means known in the art. For example, DNA clones for microinjection can be cleaved with enzymes appropriate for removing the bacterial plasmid sequences, and the DNA fragments electrophoresed on 1% agarose gels in TBE buffer, using standard techniques. The DNA bands are visualized by staining with ethidium bromide, and the band containing the expression sequences is excised. The excised band is then placed in dialysis bags containing 0.3 M sodium acetate, pH 7.0. DNA is electroeluted into the dialysis bags, extracted with a 1:1 phenol:chloroform solution and precipitated by two volumes of ethanol. The DNA is redissolved in 1 ml of low salt buffer (0.2 M NaCl, 20 mM Tris, pH 7.4, and 1 mM EDTA) and purified on an Elutip-D™ column. The column is first primed with 3 ml of high salt buffer (1 M NaCl, 20 mM Tris, pH 7.4, and 1 mM EDTA) followed by washing with 5 ml of low salt buffer. The DNA solutions are passed through the column three times to bind DNA to the column matrix. After one wash with 3 ml of low salt buffer, the DNA is eluted with 0.4 ml high salt buffer and precipitated by two volumes of ethanol. DNA concentrations are measured by absorption at 260 nm in a UV spectrophotometer. For microinjection, DNA concentrations are adjusted to 3 μg/ml in 5 mM Tris, pH 7.4 and 0.1 mM EDTA. Other methods for purification of DNA for microinjection are described in in Palmiter et al. (1982); and in Sambrook et al. (2001).

In an exemplary microinjection procedure, female mice six weeks of age are induced to superovulate with a 5 IU injection (0.1 cc, ip) of pregnant mare serum gonadotropin (PMSG; Sigma) followed 48 hours later by a 5 IU injection (0.1 cc, ip) of human chorionic gonadotropin (hCG; Sigma). Females are placed with males immediately after hCG injection. Twenty-one hours after hCG injection, the mated females are sacrificed by C02 asphyxiation or cervical dislocation and embryos are recovered from excised oviducts and placed in Dulbecco's phosphate buffered saline with 0.5% bovine serum albumin (BSA; Sigma). Surrounding cumulus cells are removed with hyaluronidase (1 mg/ml). Pronuclear embryos are then washed and placed in Earle's balanced salt solution containing 0.5% BSA (EBSS) in a 37.5° C. incubator with a humidified atmosphere at 5% CO₂, 95% air until the time of injection. Embryos can be implanted at the two-cell stage.

Randomly cycling adult female mice are paired with vasectomized males. C57BL/6 or Swiss mice or other comparable strains can be used for this purpose. Recipient females are mated at the same time as donor females. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmakers forceps. Embryos to be transferred are placed in DPBS (Dulbecco's phosphate buffered saline) and in the tip of a transfer pipet (about 10 to 12 embryos). The pipet tip is inserted into the infundibulum and the embryos transferred. After the transfer, the incision is closed by two sutures.

### VI. DEFINITIONS

As used herein, the term “heart failure” is broadly used to mean any condition that reduces the ability of the heart to pump blood. As a result, congestion and edema develop in the tissues. Most frequently, heart failure is caused by decreased contractility of the myocardium, resulting from reduced coronary blood flow; however, many other factors may result in heart failure, including damage to the heart valves, vitamin deficiency, and primary cardiac muscle disease. Though the precise physiological mechanisms of heart failure are not entirely understood, heart failure is generally believed to involve disorders in several cardiac autonomic properties, including sympathetic, parasympathetic, and baroreceptor responses. The phrase “manifestations of heart failure” is used broadly to encompass all of the sequelae associated with heart failure, such as shortness of breath, pitting edema, an enlarged tender liver, engorged neck veins, pulmonary rales and the like including laboratory findings associated with heart failure.

The term “treatment” or grammatical equivalents encompasses the improvement and/or reversal of the symptoms of heart failure (i.e., the ability of the heart to pump blood). “Improvement in the physiologic function” of the heart may be assessed using any of the measurements described herein (e.g., measurement of ejection fraction, fractional shortening, left ventricular internal dimension, heart rate, etc.), as well as any effect upon the animal's survival. In use of animal models, the response of treated transgenic animals and untreated transgenic animals is compared using any of the assays described herein (in addition, treated and untreated non-transgenic animals may be included as controls). A compound which causes an improvement in any parameter associated with heart failure used in the screening methods of the instant invention may thereby be identified as a therapeutic compound.

The term “dilated cardiomyopathy” refers to a type of heart failure characterized by the presence of a symmetrically dilated left ventricle with poor systolic contractile function and, in addition, frequently involves the right ventricle.

The term “compound” refers to any chemical entity, pharmaceutical, drug, and the like that can be used to treat or prevent a disease, illness, sickness, or disorder of bodily function. Compounds comprise both known and potential therapeutic compounds. A compound can be determined to be therapeutic by screening using the screening methods of the present invention. A “known therapeutic compound” refers to a therapeutic compound that has been shown (e.g., through animal trials or prior experience with administration to humans) to be effective in such treatment. In other words, a known therapeutic compound is not limited to a compound efficacious in the treatment of heart failure.

As used herein, the term “agonist” refers to molecules or compounds which mimic the action of a “native” or “natural” compound. Agonists may be homologous to these natural compounds in respect to conformation, charge or other characteristics. Thus, agonists may be recognized by receptors expressed on cell surfaces. This recognition may result in physiologic and/or biochemical changes within the cell, such that the cell reacts to the presence of the agonist in the same manner as if the natural compound was present. Agonists may include proteins, nucleic acids, carbohydrates, or any other molecules that interact with a molecule, receptor, and/or pathway of interest.

As used herein, the term “cardiac hypertrophy” refers to the process in which adult cardiac myocytes respond to stress through hypertrophic growth. Such growth is characterized by cell size increases without cell division, assembling of additional sarcomeres within the cell to maximize force generation, and an activation of a fetal cardiac gene program. Cardiac hypertrophy is often associated with increased risk of morbidity and mortality, and thus studies aimed at understanding the molecular mechanisms of cardiac hypertrophy could have a significant impact on human health.

As used herein, the terms “antagonist” and “inhibitor” refer to molecules or compounds which inhibit the action of a cellular factor that may be involved in cardiac hypertrophy. Antagonists may or may not be homologous to these natural compounds in respect to conformation, charge or other characteristics. Thus, antagonists may be recognized by the same or different receptors that are recognized by an agonist. Antagonists may have allosteric effects which prevent the action of an agonist. Alternatively, antagonists may prevent the function of the agonist. In contrast to the agonists, antagonistic compounds do not result in pathologic and/or biochemical changes within the cell such that the cell reacts to the presence of the antagonist in the same manner as if the cellular factor was present. Antagonists and inhibitors may include proteins, nucleic acids, carbohydrates, or any other molecules which bind or interact with a receptor, molecule, and/or pathway of interest.

As used herein, the term “modulate” refers to a change or an alteration in the biological activity. Modulation may be an increase or a decrease in protein activity, a change in binding characteristics, or any other change in the biological, functional, or immunological properties associated with the activity of a protein or other structure of interest. The term “modulator” refers to any molecule or compound which is capable of changing or altering biological activity as described above.

The term “β-adrenergic receptor antagonist” refers to a chemical compound or entity that is capable of blocking, either partially or completely, the beta (β) type of adrenoreceptors (i.e., receptors of the adrenergic system that respond to catecholamines, especially norepinephrine). Some β-adrenergic receptor antagonists exhibit a degree of specificity for one receptor subtype (generally β₁); such antagonists are termed “β₁-specific adrenergic receptor antagonists” and “β₂-specific adrenergic receptor antagonists.” The term β-adrenergic receptor antagonist” refers to chemical compounds that are selective and non-selective antagonists. Examples of β-adrenergic receptor antagonists include, but are not limited to, acebutolol, atenolol, butoxamine, carteolol, esmolol, labetolol, metoprolol, nadolol, penbutolol, propanolol, and timolol. The use of derivatives of known β-adrenergic receptor antagonists is encompassed by the methods of the present invention. Indeed any compound, which functionally behaves as a β-adrenergic receptor antagonist is encompassed by the methods of the present invention.

The terms “angiotensin-converting enzyme inhibitor” or “ACE inhibitor” refer to a chemical compound or entity that is capable of inhibiting, either partially or completely, the enzyme involved in the conversion of the relatively inactive angiotensin I to the active angiotensin II in the rennin-angiotensin system. In addition, the ACE inhibitors concomitantly inhibit the degradation of bradykinin, which likely significantly enhances the antihypertensive effect of the ACE inhibitors. Examples of ACE inhibitors include, but are not limited to, benazepril, captopril, enalopril, fosinopril, lisinopril, quiapril and ramipril. The use of derivatives of known ACE inhibitors is encompassed by the methods of the present invention. Indeed any compound, which functionally behaves as an ACE inhibitor, is encompassed by the methods of the present invention.

As used herein, the term “genotypes” refers to the actual genetic make-up of an organism, while “phenotype” refers to physical traits displayed by an individual. In addition, the “phenotype” is the result of selective expression of the genome (i.e., it is an expression of the cell history and its response to the extracellular environment). Indeed, the human genome contains an estimated 30,000-35,000 genes. In each cell type, only a small (i.e., 10-15%) fraction of these genes are expressed.

### VII. EXAMPLES

The following examples are included to further illustrate various aspects of the invention. It should be appreciated by those of skill in the art that the techniques disclosed in the examples which follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Generation of a conditional HDAC3 allele. An HDAC3 targeting vector was generated using the pGKNEO-F2L2-DTA vector. This vector contains a neomycin resistance cassette flanked by frt and loxP sites and a diphtheria toxin gene cassette. The arms for homologous recombination were generated by high fidelity PCR amplification (Roche Expand High Fidelity) of 129SvEv genomic DNA. The targeting vector was linearized with PvuI and electroporated into 129SvEv-derived ES cells. Five hundred ES cell clones were screened for homologous recombination by Southern blot analysis. Genomic DNA was digested with BamHI and successful loxP site incorporation was confirmed with both a 5′ and 3′ probe. Targeted ES cells were injected into the blastocysts of C57BL/6 females to generate chimeric mice. Chimeras were bred to C57BL/6 females to achieve germline transmission.

Histology, immunohistochemistry, and electron microscopy. Tissues were fixed in 4% paraformaldehyde, embedded in paraffin, and sectioned at 5 μm intervals. Sections were stained with hematoxylin and eosin or Masson's trichrome using standard procedures. For neutral lipid staining, hearts were fixed in 4% paraformaldehyde, cryo-embedded, and stained with Oil Red O and counterstained with hematoxylin. For transmission electron microscopy, left ventricular tissue was minced and fixed in 2% paraformaldehyde, 2.5% glutaraldehyde, 0.1M cacodylate buffer, prepared according to standard protocol and performed at the UT Southwestern Molecular and Cellular Imaging Facility using a Tecnai G2 Spirit 120 KV TEM.

RT-PCR and microarray. For all gene expression analyses from wild-type and HDAC3cKO mice, 4 wild-type and 4 HDAC3cKO hearts were isolated and analyzed. Total RNA was purified using TRIzol reagent according to manufacturer's instructions. For RT-PCR, total RNA was used for reverse transcriptase using random hexamer primers. Primer sequences are available upon request. Quantitative real time PCR was performed using Taqman probes purchased from ABI. For microarray, RNA was extracted from either 3 wild-type or HDAC3cKO hearts and subsequently pooled prior to analysis. Microarray analysis was performed using the Mouse Genome 430 2.0 Array (Affymetrix). All heart RNA was from ventricle tissue only. Microarray analysis was performed by the UT Southwestern Microarray Core Facility using the Mouse Genome 430 2.0 Array (Affymetrix) as described (Davis et al., 2006)

ChIP. Neonatal rat myocytes were prepared as described (Antos et al., 2003). After 24 hrs induction, chromatin was harvested as described (Nelson et al., 2006). Briefly, cells were formaldehyde cross-linked and lysed, and chromatin was sheared by sonication to ˜500-base-pair (bp) fragments. Sheared chromatin was immunoprecipitated with antibodies against HA (Sigma), HDAC3 (Abcam), or PPARα (Santa Cruz) and DNA was isolated and analyzed by PCR with primers flanking binding sites for the indicated response element. Primers are available upon request. For quantitative ChIP, myocytes were isolated from wild-type and HDAC3cKO hearts and chromatin was isolated as described. Sheared chromatin was immunoprecipitated with Acetyl-H3 (Upstate) and DNA was analyzed as before. Error bars represent each reaction performed in triplicate. Similar results were obtained when experiment was repeated.

Echocardiography. Cardiac function was analyzed by 2-dimensional echocardiography on nonsedated mice using a Vingmed System (GE Vingmed Ultrasound) and a 11.5-MHz linear array transducer. M-mode tracings were used to measure anterior and posterior wall thicknesses at end diastole and end systole. LV internal diameter (LVID) was measured as the largest anteroposterior diameter in either diastole (LVIDd) or systole (LVIDs). A single observer blinded to mouse genotypes analyzed the data. LV FS was calculated according to the following formula: FS (%)=[(LVIDd−LVIDs)/LVIDd]×100.

Electrocardiography (ECG). ECG was performed on sedated adult mice using Accutac Diaphoretic ECG Electrodes (ConMed Corp). Pads were attached to all four limbs, and leads I, II, III, aVR, aVL and aVF were recorded using PageWriter XLs (Hewlett Packard). Traces were recorded using identical settings between wild-type and HDAC3cKO mice (50 mm/sec; 20 mm/mV).

Western blotting and histone isolation. Heart tissue was homogenized in lysis buffer (50 mM Tris at pH 7.4, 150 mM NaCl, 1% Triton X-100, 1 mM EDTA) supplemented with protease inhibitors (Complete Mini, EDTA-free, Roche) and centrifuged at 14,000 g for 5 min, and supernatent was recovered. Ten μg of protein was resolved by SDS-PAGE on a 10% acrylamide gel and analyzed by Western blot using antibodies against HDAC3 (rabbit polyclonal, 1:1000, Abcam) or eIF5 (rabbit polyclonal, 1:1000, Santa Cruz Biotechnology) as a loading control.

Histones were extracted from heart tissues using standard procedures. Briefly, heart tissue was disrupted with a pestel in PBS containing 0.5% Triton-X-100, 1 mM PMSF and 3 μM TSA. Nuclei were pelleted, resuspended in 0.4 N H₂SO₄ and extracted for 2 h at 4° C. Histones were precipitated by adding 10× ice-cold acetone, incubating at −20° C. for 2 hours and after centrifugation were resuspended in water by sonification. Western blot on isolated histones was performed with the following antibodies: Acetyl-H3 (Upstate), Acetyl-H4 (Upstate), poly-acetyl-lysine (Cell Signaling), and histone H3 (Cell Signaling).

Myocardial triglyceride levels. Lipids were extracted from ventricular tissue using a modified Bligh and Dyer technique. Briefly, tissue was homogenized in an ice-cold chloroform/methanol/water (2:1:0.8) solution. Additional chloroform and water was added to separate layers and the mixture was centrifuged at 12,000×g. Following centrifugation, the chloroform layer was extracted, evaporated, and the resultant residue was resuspended in 0.5 ml isopropanol. Triglyceride content was quantified using a triglyceride quantification kit (Sigma).

Animal studies. For fasting studies, male HDAC3cKO and wild-type littermates were fasted for 24 hrs. After 24 hrs, mice were sacrificed, hearts extracted, and prepared for Oil Red O staining. Control mice were allowed standard chow ad libitum. Wy14,643 (Biomol) (50 mg/kg body weight) in 50% DMSO/saline was administered as a single intraperitoneal injection. Control littermates were administered vehicle and hearts were extracted 8 hrs after injection. All animal studies were approved by the IACUC of the University of Texas Southwestern Medical Center.

Mitochondrial function assays. Eight week-old wild-type and HDAC3cKO hearts were perfused in mannitol sucrose buffer and snap frozen under liquid nitrogen. Mitochondrial function assays were performed as described (Yu et al., 2007).

Statistical methods. Values are presented as ±SEM unless otherwise noted. Gene expression was normalized to 18S Ribosomal RNA and calculated as relative change. Statistics were calculated with Excel. A p-value of <0.05 was considered to be statistically significant.

### Example 2

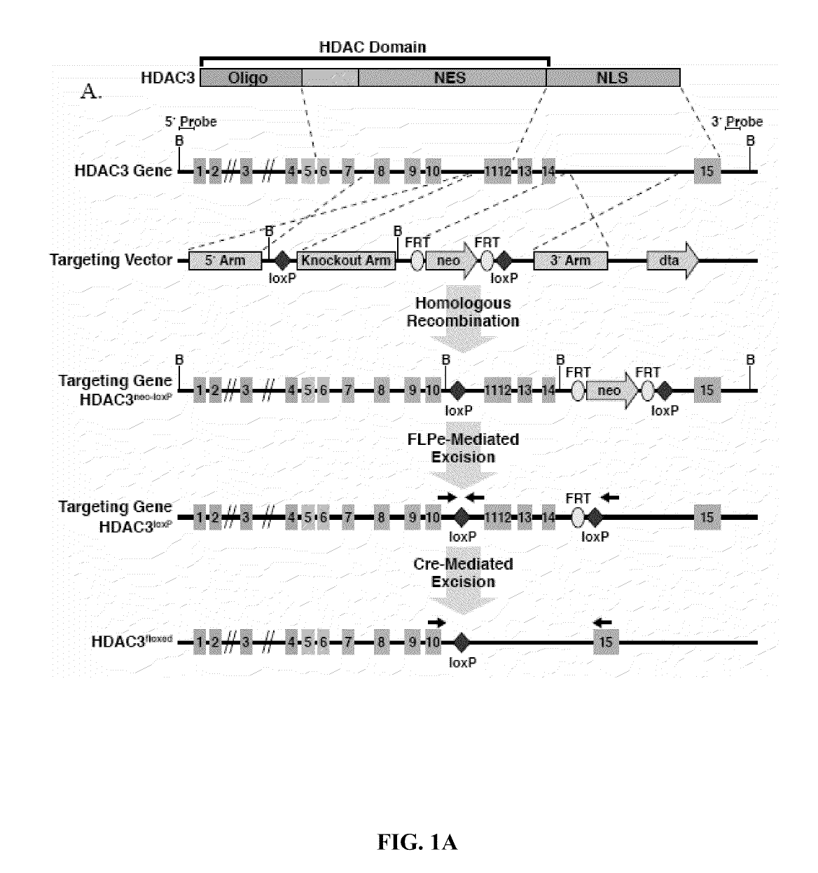

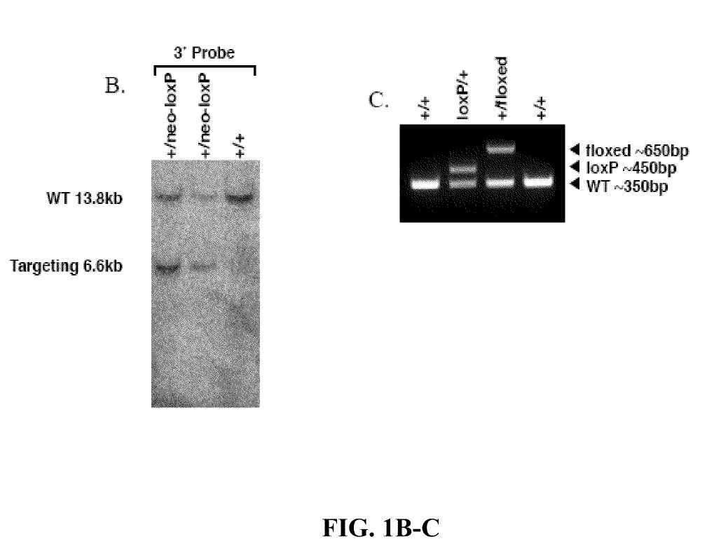

Conditional deletion of HDAC3. Given the embryonic and neonatal lethality associated with loss of HDAC1 or HDAC2, the inventors generated a conditional null allele of HDAC3 to investigate the role of HDAC3 in the adult heart. Targeting of HDAC3 was performed by introducing loxP sites upstream of exon 11 and downstream of exon 14 through homologous recombination (FIG. 1A). This mutation deletes almost all of the nuclear import sequence and a carboxy-terminal region that is necessary for transcriptional repression (Guenther et al., 2001). Germline transmission was detected by Southern blot and deletion of HDAC3 was confirmed at the genomic level (FIG. 1C). HDAC3^{neo-loxP} mice were bred to CAG-Cre (Sakai and Miyazaki, 1997) transgenic mice, which express Cre recombinase ubiquitously, allowing for the generation of HDAC3^{+/-} mice. HDAC3^{+/-} mice were intercrossed to obtain HDAC3^{-/-} mice, which died before E9.5 due to defects in gastrulation (data not shown).

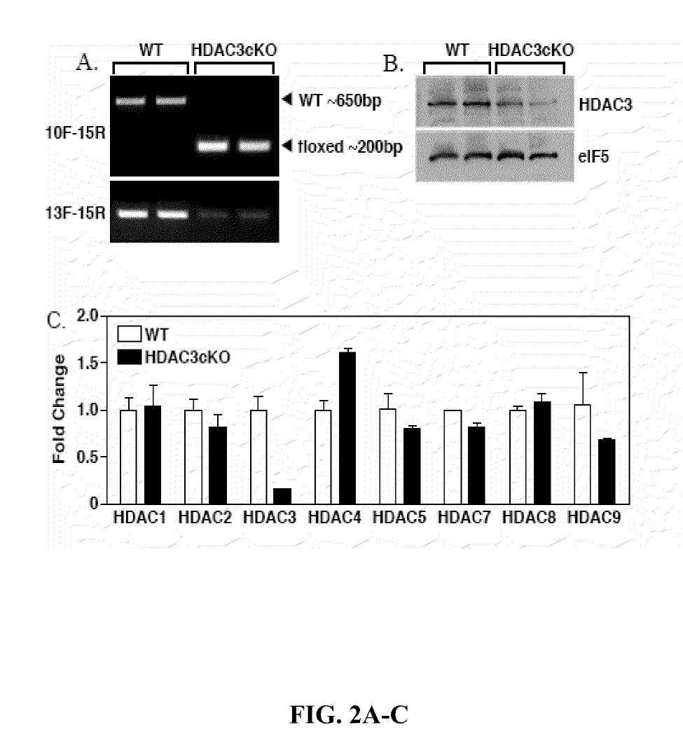

Cardiac deletion of HDAC3 causes cardiac hypertrophy. To circumvent embryonic lethality, the inventors deleted HDAC3 specifically in the heart by breeding homozygous HDAC3^{loxP/loxP} mice to transgenic mice expressing Cre recombinase under the control of the α-myosin heavy chain (αMHC) promoter (Agah et al., 1997). Cardiomyocyte-restricted deletion of HDAC3 (hereafter referred to as HDAC3cKO for HDAC3 cardiac knock-out, and wild-type represents HDAC3^{loxP/loxP} mice) was confirmed by RT-PCR and Western blot (FIGS. 2A-B). RT-PCR of HDAC3 using primers flanking the foxed region showed efficient deletion of HDAC3, however, residual expression of HDAC3 was seen using primers within the deleted region as well as by Western blot, suggesting HDAC3 is very lowly expressed in αMHC-Cre negative cell-types such as cardiac fibroblasts (FIGS. 2A-B). Expression levels of other class I and class II HDACs were not significantly altered in HDAC3cKO hearts (FIG. 2C).

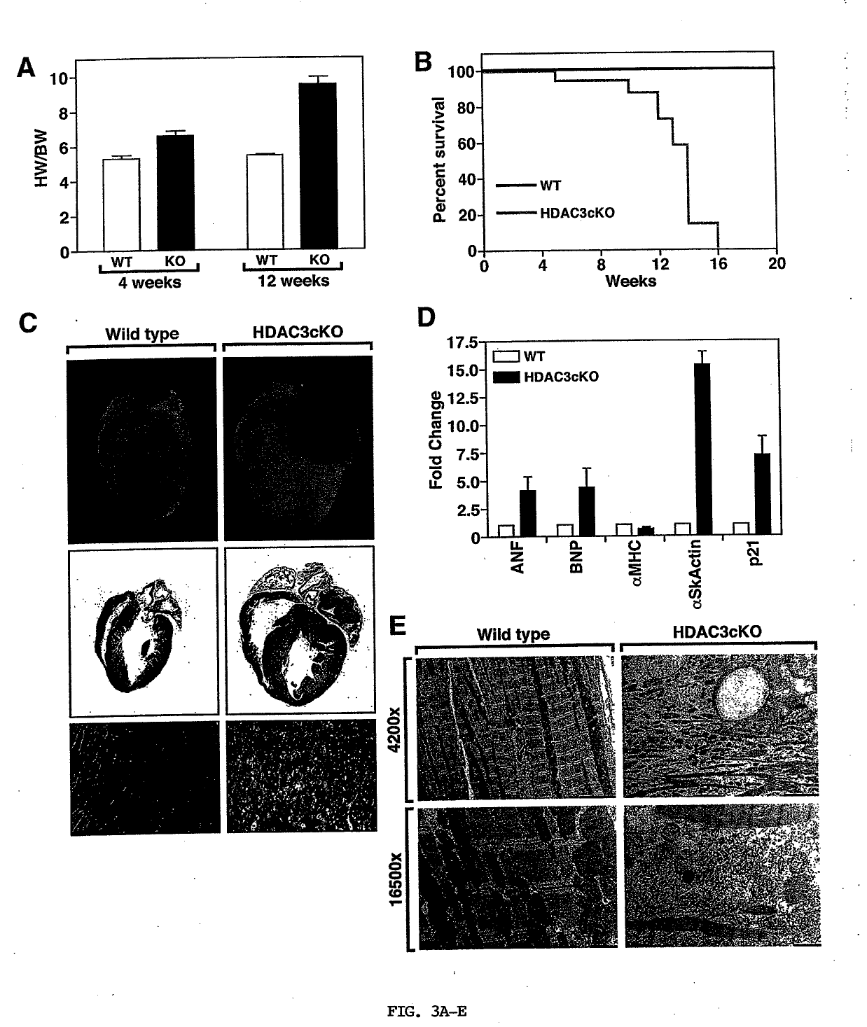

HDAC3cKO mice were born at Mendelian ratios, however, signs of cardiac hypertrophy, assessed by heart weight to body weight (HW/BW) ratios, were apparent by 4 weeks of age and were exacerbated by 12 weeks of age, resulting a 72% increase in HW/BW ratio compared to wild-type littermates (FIG. 3A). Cardiac deletion of HDAC3 resulted in 100% lethality by 16 weeks of age, with significant lethality occurring between 12 and 14 weeks (FIG. 3B). Hearts of HDAC3cKO mice were hypertrophic and showed enlargement of both right and left atria (FIG. 3C). Histology confirmed cardiomyocyte hypertrophy, especially in the left ventricular free wall and septum, as well as robust interstitial fibrosis in HDAC3cKO mice compared to wild-type littermates (FIG. 3C). Cardiac stress markers atrial natriuretic factor (ANF, Nppa), brain natriuretic peptide (BNP, Nppb), and α-skeletal actin (Acta1) were significantly up-regulated as early as 8 weeks of age in mutant mice, consistent with the hypertrophy seen by histology (FIG. 3D). Expression of p21 (Cdkn1a), shown to be repressed by class I HDACs in a variety of cell types (Gui et al., 2004), was also significantly up-regulated in hearts of HDAC3cKO mice, supporting the role of class I HDACs as transcriptional repressors of p21.

Ultrastructural analysis of the left ventricular free wall of the adult myocardium revealed that the normal juxtaposition of sarcomeres to mitochondria (FIG. 3E), which facilitates efficient myofibrillar contraction and relaxation in normal cardiomyocytes, was aberrant in HDAC3cKO mutants. Instead, cardiac deletion of HDAC3 resulted in disorganized and fragmented myofibrils, associated with intracellular debris and disarrangement of mitochondria that showed reduced cristae density (FIG. 3E).

To determine if there is a correlation between HDAC3 expression levels and pathological conditions of the heart, the inventors examined HDAC3 expression in multiple settings of hypertrophy and failure. HDAC3 levels were not significantly altered following angiotensin infusion, aortic banding, myocardial infarction, or in the Zucker Diabetic Fatty (ZDF) rat heart, however, HDAC3 expression was decreased following isoproterenol infusion (data not shown).

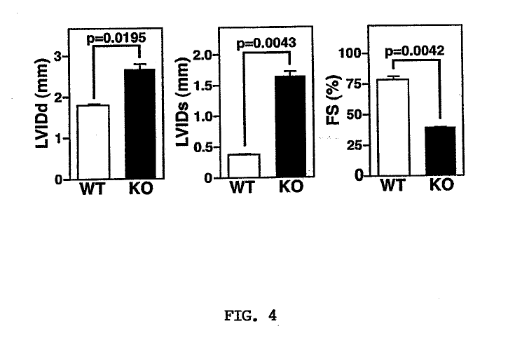

Functional analyses of wild-type and HDAC3cKO mice were performed at 12 weeks of age by echocardiography. As shown in FIG. 4, HDAC3cKO mice showed diminished contractility and ventricular dysfunction as indicated by reduced fractional shortening (39.25±0.75 vs. 78.65±4.35 for WT) and increased left ventricular chamber dilatation as assessed by systolic and diastolic internal diameters, LVIDs and LVIDd, respectively. Additionally, electrocardiography (ECG) was performed on 8-week and 14-week-old mice to determine if HDAC3cKO mice have conduction system defects. HDAC3cKO mice showed no overt abnormalities in their sinus rhythm compared to wild-type littermates (data not shown). Furthermore, continuous telemetry was performed on wild-type and HDAC3cKO mice from 12 to 16 weeks of age to determine if arrhythmias contribute to the sudden death. No signs of cardiac arrhythmia were observed in HDAC3cKO when compared to wild-type littermates (data not shown).

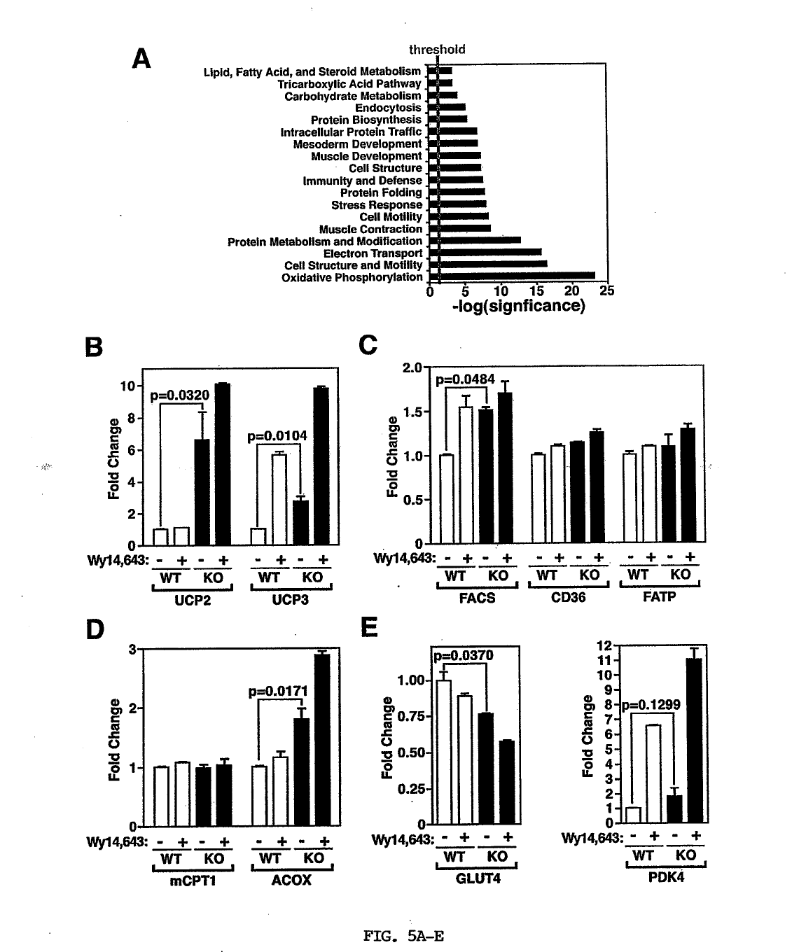

Up-regulation of myocardial energetic genes from cardiac deletion of HDAC3. In an effort to more precisely understand the primary cause of cardiomyopathy in HDAC3 mutant hearts, the inventors performed microarray analysis on left ventricles from 5-week-old mice. At this timepoint, mutant hearts showed moderate increases in HW/BW ratios and relatively minor changes in cardiac stress markers (FIG. 3A and data not shown). Gene ontology analysis of significantly up-regulated transcripts in HDAC3cKO mice revealed dramatic dysregulation of cardiac metabolism in the mutant hearts (FIG. 5A and data not shown).

Cardiac energetics is tightly regulated by the peroxisome proliferator-activated receptor (PPAR) and the estrogen-related receptor (ERR) families of nuclear hormone receptors (Huss and Kelly, 2004), and PPARα cardiac overexpression results in diabetic cardiomyopathy (Finck et al., 2002). Expression levels for PPARα, PPARα, ERRα, and PGC-1α were unchanged in HDAC3cKO hearts compared to wild-type littermates, suggesting the phenotype is independent of changes in receptor or coactivator expression (data not shown). To determine if the cardiac hypertrophy and ventricular dysfunction in HDAC3 mutant mice resulted from rampant nuclear receptor-dependent gene activation, the inventors assayed known PPARα target genes in ventricles of HDAC3cKO mice. PPARα has been shown to regulate expression of the mitochondrial uncoupling proteins, UCP2 and UCP3 (Young et al., 2001). Accordingly, transcript levels for both UCP2 and UCP3 were significantly up-regulated at baseline (6.5-fold and 2.9-fold, respectively) and this induction was increased upon administration of Wy14,643, a synthetic PPARα-agonist (FIG. 5B).

Real-time PCR analysis of genes encoding fatty acid import, transport, and esterification (fatty acid transport protein [FATP, Slc27a1], CD36, and fatty acyl-CoA synthetase [FACS, Acsl1]) showed modest to insignificant changes at baseline. Following administration of Wy14,643, these levels were up-regulated (FIG. 5C), consistent with previous studies showing PPAR ligands to be rate-limiting under physiological conditions (Finck et al., 2002).

The expression of PPARα-responsive genes involved in fatty acid oxidation was also analyzed by real time PCR. Similar to the expression of PPARα dependent genes involved in fatty acid import, induction of PPARα-responsive genes involved in fatty acid oxidation was modest at baseline. Surprisingly, expression levels of muscle carnitine palmitoyl transferase-1 (mCPT1) were unchanged in HDAC3cKO mice compared to wild-type mice and Wy14,643 treatment had no effect on mCPT-1 transcript levels in wild-type or HDAC3cKO hearts (FIG. 5D), however additional enzymes involved in mitochondrial fatty acid oxidation were significantly up-regulated at baseline, including long- and very long-chain acyl-Coenzyme A dehydrogenase (LCAD, Acadl and VLCAD, Acadvl, respectively) (data not shown). Conversely, acyl-CoA oxidase 1 (ACOX) was significantly increased in HDAC3cKO hearts and was further up-regulated in response to Wy14,643 (FIG. 5D). ACOX is the first enzyme in peroxisomal fatty acid β-oxidation, suggesting HDAC3cKO hearts possess greater fatty acid oxidation potential than wild-type littermates.

Decreased expression of genes involved in glucose utilization from cardiac deletion of HDAC3. In diabetic cardiomyopathies, increased expression of genes involved in fatty acid import and fatty acid oxidation is coupled to a decreased utilization of the glucose oxidation pathway (Stanley et al., 1997). To determine if HDAC3cKO mice show defects in glucose uptake and utilization, the inventors examined expression levels of the glucose transporters, GLUT1 and GLUT4. GLUT1 levels were relatively unchanged in HDAC3cKO mice compared to wild-type (data not shown), whereas GLUT4 expression was significantly down-regulated in HDAC3cKO mice (FIG. 5E) and was further decreased in response to Wy14,643 (FIG. 5E). GLUT1 controls basal glucose uptake while GLUT4 regulates glucose transport in an insulin-sensitive dependent manner. Down-regulation of GLUT4 in HDAC3cKO mice is consistent with the phenotype resulting from excessive PPARα activity.

Pyruvate dehydrogenase kinase 4 (PDK4) regulates the pyruvate dehydrogenase (PDH) complex through phosphorylation and subsequent inactivation. PDK4 levels and activity are increased in diabetic hearts (Glyn-Jones et al., 2007). Similarly, HDAC3cKO mice showed an increase in expression of PDK4 that was further elevated upon administration of Wy14,643 (FIG. 5E).

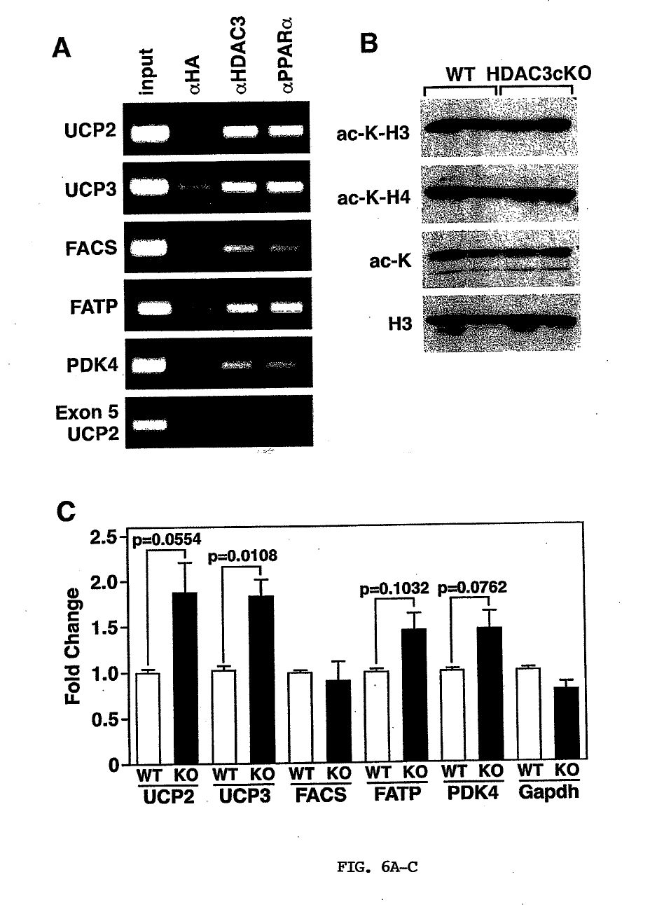

Localization of HDAC3 to the promoters of PPARα-responsive genes. To further investigate the regulation of PPARα-responsive genes by HDAC3, the inventor performed chromatin immunoprecipitation (ChIP) on multiple genes up-regulated in HDAC3cKO hearts from neonatal rat myocytes. Immunoprecipitation of HDAC3 or PPARα was able to robustly pull down the PPAR-response element within the promoters of the UCP2, UCP3, FACS, FATP, and PDK4 genes compared to the negative anti-HA control or the negative exon 5 of UCP2 control (FIG. 6A). These findings indicate that HDAC3 resides in a repressive complex with PPARα under basal physiological conditions.

Histone acetylation is locally increased in HDAC3cKO hearts. To determine if loss of HDAC3 results in global changes in histone acetylation, the inventors isolated histones from wild-type and HDAC3cKO hearts and examined the acetylation states of histone H3 and H4. Western blot analyses for acetyl-H3, acetyl-H4, and pan-acetyl-lysine revealed no global changes in histone acetylation in HDAC3cKO hearts (FIG. 6B). The localization of HDAC3 to the promoters of multiple PPAR-responsive genes suggested changes in histone acetylation might occur locally at up-regulated transcripts. To investigate this, the inventors isolated cardiomyocytes from wild-type and HDAC3cKO mice and performed quantitative chromatin immunoprecipitation assays. Immunoprecipitation on the PPAR-responsive elements with acetyl-H3 revealed increased local acetylation levels at the promoters of multiple PPAR-responsive genes in HDAC3cKO myocytes compared to wild-type controls, but not on the promoters of genes unaltered in HDAC3cKO, such as Gapdh (FIG. 6C).

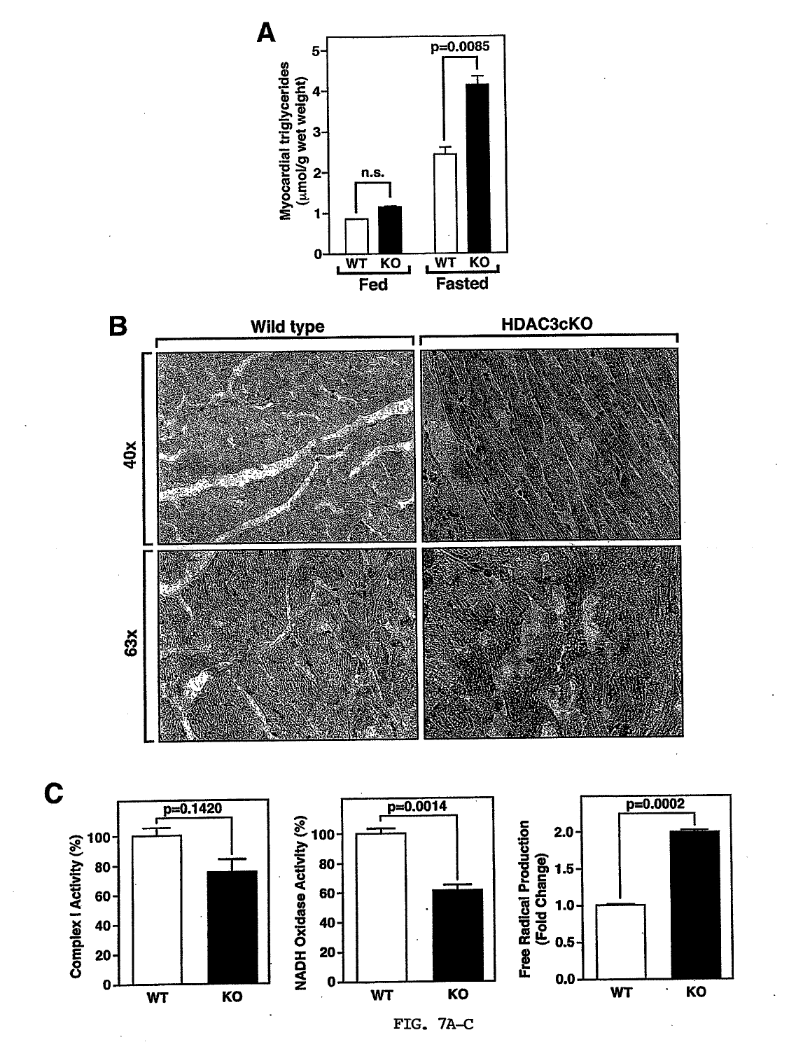

Cardiac deletion of HDAC3 causes ligand-induced lipid accumulation. The ligand-inducible activation of multiple PPAR-responsive genes in cardiomyocytes of HDAC3cKO mice suggests these mice are sensitive to increased fatty acids in the circulation. A hallmark of the diabetic heart is lipid accumulation in myocytes due to augmented fatty acid import (Murthy and Shipp, 1977). To determine if HDAC3 mutant mice show myocardial lipid accumulation following increases in circulating fatty acids, the inventors subjected HDAC3cKO and wild-type mice to a 24-hour fast, which induces circulating fatty acids that can subsequently serve as ligands for PPARs. After fasting, hearts were excised and triglycerides were quantified. Mice fed ad libitum showed no significant difference in triglyceride content between HDAC3cKO and wild-type mice (FIG. 7A). However, HDAC3cKO fasted mice showed a dramatic increase in myocardial triglycerides compared to wild-type controls (FIG. 7A). Quantification of serum fatty acid and triglyceride levels showed there to be no significant difference between wild-type and HDAC3cKO mice (data not shown). Consistent with these findings, Oil Red O staining of histological sections from fed and fasted wild-type and HDAC3cKO hearts showed no significant staining in fed mice of either genotype (data not shown), whereas fasted myocytes from HDAC3cKO mice showed a pronounced increase in neutral lipid accumulation (FIG. 7B). Neutral lipids could be readily visualized throughout both ventricular free walls as well as the ventricular septum, however the atria remained free of lipids. These results further point to HDAC3 as an important regulator of PPAR and other transcription factors that govern myocardial energy metabolism.

Mitochondrial dysfunction resulting from cardiac deletion of HDAC3. To further define the metabolic abnormalities resulting from cardiac specific deletion of HDAC3, the inventors compared mitochondrial function in wild-type and HDAC3cKO mice. Mitochondrial dysfunction is intimately linked to diabetic cardiomyopathy through defects in glucose utilization and increased fatty acid oxidation (Boudina and Abel, 2007). Increased fatty acid oxidation results in increased reducing equivalents to the electron transport chain, which in turn generates free radicals and leads to mitochondrial uncoupling (Boudina and Abel, 2006). Free fatty acids are able to directly promote free radical production through the inhibition of complex I of the electron transport chain (Cocco et al., 1999; Loskovich et al., 2005; Schonfeld and reiser, 2006). Consistently, HDAC3cKO mice showed a 25% reduction in complex I activity accompanied by a 39% reduction in NADH oxidase activity (FIG. 7C). Additionally, free radical production was increased two-fold in HDAC3cKO mice compared to wild-type littermates (FIG. 7C).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VIII. REFERENCES

The following references, to the extent that they provide exemplary procedural or other details supplementary to those set forth herein, are specifically incorporated herein by reference:
U.S. Pat. No. 4,873,191
Agah et al., J. Clin. Invest., 100:169-179, 1997.
Antos et al., J. Biol. Chem., 278:28930-28937, 2003.
Boudina and Abel, Circulation, 115:3213-3223, 2007.
Boudina and Abel, Physiology (Bethesda), 21:250-258, 2006.
Brinster et al., Proc. Natl. Acad. Sci. USA, 82(13):4438-4442, 1985.
Burkart et al., J. Clin. Invest., 117:3930-3939, 2007.
Cheng et al., Nat. Med., 10:1245-1250, 2004.
Cocco et al., Free Radic. Biol. Med., 27:51-59, 1999.
Davis et al., Mol. Cell. Biol., 26:2626-2636, 2006.
Devereux et al., Jama, 292:2350-2356, 2004.
Durand et al., Ann. Med., 27:311-317, 1995.
Fajas et al., Dev. Cell, 3:903-910, 2002.
Finck et al., J. Clin. Invest., 109:121-130, 2002.
Fischle et al., Mol. Cell, 9:45-57, 2002.
Frey and Olson, Annu. Rev. Physiol., 65:45-79, 2003.
Gardin and Lauer, Jama, 292:2396-2398, 2004.
Glyn-Jones et al., Physiol. Genomics, 28:284-293, 2007.
Gregoire et al., Mol. Cell. Biol., 27:1280-1295, 2007.
Grozinger and Schreiber, Chem. Biol., 9:3-16, 2002.
Grozinger et al., Proc. Natl. Acad. Sci. USA, 96:4868-4873, 1999.
Guan et al., Genes Dev., 19:453-461, 2005.
Guenther et al., Mol. Cell. Biol., 21:6091-6101, 2001.
Gui et al., Proc. Natl. Acad. Sci. USA, 101:1241-1246, 2004.
Hill and Olson, N. Engl. J. Med., 358:1370-1380, 2008.
Huss and Kelly, Circ. Res., 95:568-578, 2004.
Huss et al., Mol. Cell. Biol., 24:9079-9091, 2004.
Jenuwein and Allis, Science, 293:1074-1080, 2001.
Kao et al., Genes Dev., 14:55-66, 2000.
Kee et al., Circulation, 113:51-59, 2006.
Kersten et al., J. Clin. Invest., 103:1489-1498, 1999.
Kim et al., J. Clin. Invest., 118:124-132, 2008.
Kong et al., Circulation, 113:2579-2588, 2006.
Loskovich et al., Biochem. J, 387:677-683, 2005.
McKinsey and Olson, J. Clin. Invest., 115:538-546, 2005.
McKinsey and Olson, Trends Genet., 20:206-213, 2004.
McKinsey et al., Nature, 408:106-111, 2000.
Methot et al., Bioorg. Med. Chem. Lett., 18(3):973-978, 2008.
Montgomery et al., Genes Dev., 21:1790-1802, 2007.
Moradei et al., J. Med. Chem., 50(23):5543-5546, 2007.
Murthy and Shipp, Diabetes, 26:222-229, 1977.
Nelson et al., Nucleic Acids Res., 34:e2, 2006.
Okin et al., Jama, 292:2343-2349, 2004.
Olson and Schneider, Genes Dev., 17:1937-1956, 2003, 2003.
Palmiter et al., Cell, 29:701, 1982.
PCT Application WO 84/03564.
Remington's Pharmaceutical Sciences” 15^{th} Ed., 1035-1038 and 1570-1580, 1990.
Roth et al., Annu. Rev. Biochem., 70:81-120, 2001.
Sakai and Miyazaki, Biochem. Biophys. Res. Commun., 237:318-324, 1997.
Sambrook et al., In: Molecular cloning, Cold Spring Harbor Laboratory Press, Cold Spring Harbor, N.Y., 2001.
Schonfeld and Reiser, J. Biol. Chem., 281:7136-7142, 2006.
Stanley et al., Cardiovasc. Res., 34:25-33, 1997.
Tong et al., Nucleic Acids Res., 30:1114-23, 2002.
Trivedi et al., Nat. Med., 13:324-331, 2007.
Van den Wyngaert et al., FEBS Lett., 478:77-83, 2000.
Villagra et al., J. Biol. Chem., 282:35457-35470, 2007.
Watanabe et al., J. Biol. Chem., 275:22293-22299, 2000.
Witter et al., Bioorg. Med. Chem. Lett., 18(2):726-731, 2008.
Workman and Kingston, Annu. Rev. Biochem., 67:545-579, 1998.
Young et al., Faseb J., 15:833-845, 2001.
Young et al., Handbook of Applied Therapeutics, 7.1-7.12 and 9.1-9.10, 1989.
Yu et al., Cardiovasc. Diabetol., 6:6, 2007.
Zhang et al., Cell, 110:479-488, 2002.
Zhou et al., Proc. Natl. Acad. Sci., 98:10572-10577, 2001.

What is claimed is:

1. A method of treating pathologic cardiac hypertrophy and/or heart failure comprising:
(a) identifying a patient having pathologic cardiac hypertrophy and/or heart failure; and
(b) administering to said patient a histone deacetylase inhibitor that selectively inhibits HDAC1, HDAC2, or both HDAC1 and HDAC2, over HDAC3.

2. The method of claim 1, wherein said inhibitor is a heteroaryl substituted benzamide or biaryl benzamide, optionally substituted.

3. The method of claim 1, wherein administering comprises oral administration of said histone deacetylase inhibitor.

4. The method of claim 1, wherein administering comprises intravenous, transdermal, sustained release, suppository, or sublingual administration.

5. The method of claim 1, further comprising administering to said patient a second therapeutic regimen.

6. The method of claim 5, wherein said second therapeutic regimen is selected from the group consisting of a β blocker, an iontrope, diuretic, ACE inhibitor, All antagonist, Ca⁺⁺-blocker, nitrate, thrombolytic, and anti-platelet.

7. The method of claim 5, wherein said second therapeutic regimen is administered at the same time as said histone deacetylase inhibitor.

8. The method of claim 5, wherein said second therapeutic regimen is administered either before or after said histone deacetylase inhibitor.

9. The method of claim 1, wherein treating comprises improving one or more symptoms of pathologic cardiac hypertrophy and/or heart failure.

10. The method of claim 9, wherein said one or more symptoms comprises increased exercise capacity, increased blood ejection volume, left ventricular end diastolic pressure, pulmonary capillary wedge pressure, cardiac output, cardiac index, pulmonary artery pressures, left ventricular end systolic and diastolic dimensions, left and right ventricular wall stress, or wall tension, quality of life, disease-related morbidity and mortality.

11. A method of preventing pathologic cardiac hypertrophy and/or heart failure comprising:
(a) identifying a subject at risk of developing pathologic cardiac hypertrophy and/or heart failure; and
(b) administering to said subject a histone deacetylase inhibitor that selectively inhibits HDAC1 and/or HDAC2 over HDAC3.

12. The method of claim 11, wherein said inhibitor is a heteroaryl substituted benzamide or biaryl benzamide, optionally substituted.

13. The method of claim 11, wherein administering comprises oral administration of said histone deacetylase inhibitor.

14. The method of claim 11, wherein administering comprises intravenous, transdermal, sustained release, suppository, or sublingual administration.

15. The method of claim 11, wherein the subject at risk may exhibit one or more of long standing uncontrolled hypertension, uncorrected valvular disease, chronic angina and/or recent myocardial infarction.

16. The method of claim 11, further comprising administering to said subject a second prophylatic regimen.

17. The method of claim 16, wherein said second prophylatic regimen is selected from the group consisting of a β blocker, an iontrope, diuretic, ACE-I, All antagonist, Ca⁺⁺-blocker, nitrate, thrombolytic, and anti-platelet.

18. The method of claim 16, wherein said second prophylatic regimen is administered at the same time as said histone deacetylase inhibitor.

19. The method of claim 16, wherein said second prophylatic regimen is administered either before or after said histone deacetylase inhibitor.

20. The method of claim 11, wherein preventing comprises preventing pathological cardiac hypertrophy from developing into heart failure.

21. A method of identifying an inhibitor of pathologic cardiac hypertrophy and/or heart failure comprising:
(a) providing a histone deacetylase inhibitor;
(b) treating a myocyte with said histone deacetylase inhibitor; and
(c) measuring the activity of at least HDAC1, HDAC2 and HDAC3,
wherein a relative decrease in the activity of HDAC 1 and/or HDAC2 versus HDAC3, as compared to an untreated myocyte, identifies said histone deacetylase inhibitor as an inhibitor of pathologic cardiac hypertrophy and/or heart failure.

22. The method of claim 21, wherein said myocyte is subjected to a stimulus that triggers a hypertrophic response.

23. The method of claim 22, wherein said stimulus is expression of a transgene.

24. The method of claim 22, wherein said stimulus is treatment with a drug.

25. The method of claim 22, wherein said hypertrophic response comprises an alteration in the expression level of one or more target genes in said myocyte, wherein expression level of said one or more target genes is indicative of cardiac hypertrophy.

26. The method of claim 25, wherein said one or more target genes is selected from the group consisting of ANF, α-MyHC, β-MyHC, α-skeletal actin, SERCA, cytochrome oxidase subunit VIII, mouse T-complex protein, insulin growth factor binding protein, Tau-microtubule-associated protein, ubiquitin carboxyl-terminal hydrolase, Thy-1 cell-surface glycoprotein, or MyHC class I antigen.

27. The method of claim 21, wherein activity is assessed by measuring release of a labeled acetyl group from a histone.

28. The method of claim 21, wherein activity is assessed by measuring the expression of (i) at least one of T-type Ca²⁺ channels, L-type Ca²⁺ channels, ssTnI, and fsTn1, and (ii) at least one of a myocardial energetic gene and/or a gene involved in glucose utilization.

29. The method of claim 28, wherein measuring the expression comprises measuring expression of a reporter protein, such as luciferase, β-gal, or green fluorescent protein, operably connected to a promoter for (i) at least one of T-type Ca²⁺ channels, L-type Ca²⁺ channels, ssTnI, and fsTn1, and (ii) at least one of a myocardial energetic gene and/or a gene involved in glucose utilization.

30. The method of claim 22, wherein said hypertrophic response comprises an alteration in one or more aspects of cellular morphology.

31. The method of claim 30, wherein said one or more aspects of cellular morphology comprises sarcomere assembly, cell size, or cell contractility.

32. The method of claim 21, wherein said myocyte is an isolated myocyte.

33. The method of claim 21, wherein said myocyte is comprised in isolated intact tissue.

34. The method of claim 21, wherein said myocyte is a cardiomyocyte.

35. The method of claim 34, wherein said cardiomyocyte is located in vivo in a functioning intact heart muscle.

36. The method of claim 35, wherein said functioning intact heart muscle is subjected to a stimulus that triggers a hypertrophic response in said intact heart muscle.

37. The method of claim 36, wherein said stimulus is a pharmacologic stimulus, aortic banding, rapid cardiac pacing, induced myocardial infarction, or transgene expression.

38. The method of claim 36, wherein said hypertrophic response comprises an alteration in right ventricle ejection fraction, left ventricle ejection fraction, ventricular wall thickness, heart weight/body weight ratio, and/or cardiac weight normalization measurement.

39. The method of claim 22, wherein said hypertrophic response comprises an alteration in total protein synthesis.

40. A transgenic non-human animal, cells of which lack at least one functional allele of HDAC3.

41. The transgenic animal of claim 40, wherein said animal lacks two functional alleles of HDAC3.

42. The transgenic animal of claim 40, wherein said animal is a rat or a mouse.
